(19) United States
(12) Patent Application Publication
(10) Pub. No.: US 2025/0163392 A1
(43) Pub. Date: May 22, 2025

(54) NUCLEIC ACID-GUIDED NICKASE FUSION PROTEINS

(71) Applicant: Inscripta, Inc., Pleasanton, CA (US)

(72) Inventors: Juhan Kim, Pleasanton, CA (US); Benjamin Mijts, Pleasanton, CA (US)

(21) Appl. No.: 18/835,077

(22) PCT Filed: Feb. 2, 2023
(86) PCT No.: PCT/US23/61877

§ 371 Date: Aug. 1, 2024

Related U.S. Application Data
(60) Provisional application No. 63/421,609, filed on Nov. 2, 2022.
(60) Provisional application No. 63/306,062, filed on Feb. 2, 2022.

(51) Int. Cl. C12N 9/12; C12N 9/22; C12N 15/11

(52) U.S. Cl. CPC C12N 9/1276; C12N 9/22; C12N 15/111; C07K 2319/09; C07K 2319/85; C12N 2310/20

(57) ABSTRACT

This disclosure provides compositions and methods useful for editing a target nucleic acid molecule. This disclosure provides MAD2019-H848A variant polypeptides, reverse transcriptases, and fusion proteins and methods of using MAD2019-H848A variant polypeptides, reverse transcriptases, and fusion proteins to edit nucleic acid molecules both in vivo and in vitro.

### CROSS-REFERENCE TO RELATED APPLICATIONS AND INCORPORATION OF SEQUENCE LISTING

This application claims the benefit of U.S. Provisional Application No. 63/306,062, filed Feb. 2, 2022, and U.S. Provisional Application No. 63/421,609, filed Nov. 2, 2022, both of which are incorporated by reference in its entirety herein. A sequence listing contained in the file named “P35216WO00_SL.XML” which is 39,526 bytes (measured in MS-Windows®) and created on Feb. 2, 2023, is filed electronically herewith and incorporated by reference in its entirety.

### FIELD

This disclosure provides engineered nucleic-acid guided proteins (e.g., nickases) and fusion proteins. The provided proteins can be used to make targeted edits to nucleic acid molecules in living cells.

### BACKGROUND

The ability to make precise, targeted changes to the genome of living cells has been a long-standing goal in biomedical research and development. Recently, various nucleases have been identified that allow manipulation of gene sequences, and thus, gene function.

The identified nucleases include nucleic-acid guided nucleases and nickases derived from the nucleic-acid guided nucleases. Nickases generate single-stranded breaks rather than double-stranded breaks. The ability to cleave only a single strand of DNA can increase the versatility of nucleic acid-guided nucleases for certain editing tasks.

One editing task that makes use of a nickase is prime editing. Prime editing combines a nickase with a reverse transcriptase to create a fusion protein. The fusion protein forms a nucleoprotein complex with a prime editing guide RNA that specifies a target site to be edited and encodes the desired edit. Prime editing is capable of creating insertions, deletions, and all 12 types of point mutations.

There is a need in the art for novel nucleases and nickases derived from the novel nickases that have varied activity in cells from different organisms, that have different cutting motifs, and/or have altered enzyme fidelity. The novel polypeptides provided herein satisfy this unmet need.

### SUMMARY

In one aspect, this disclosure provides a MAD2019-H848A polypeptide.

In one aspect, this disclosure provides a MAD2019-H848A variant polypeptide comprising an amino acid sequence at least 90% identical or similar to SEQ ID NO: 1, where the MAD2019-H848A variant polypeptide comprises an alanine at position 848 according to SEQ ID NO: 1.

In one aspect, this disclosure provides a fusion protein comprising a MAD2019-H848A variant polypeptide.

In one aspect, this disclosure provides a fusion protein comprising a Tf1 reverse transcriptase comprising an amino acid sequence at least 90% identical or similar to SEQ ID NO: 13.

In one aspect, this disclosure provides a fusion protein comprising a Tf1 reverse transcriptase comprising the amino acid sequence of SEQ ID NO: 14.

In one aspect, this disclosure provides a nucleoprotein complex comprising a MAD2019-H848A variant polypeptide.

In one aspect, this disclosure provides a nucleoprotein complex comprising a fusion protein provided herein.

In one aspect, this disclosure provides a eukaryotic cell comprising a MAD2019-H848A variant polypeptide. In one aspect, this disclosure provides a eukaryotic cell comprising a fusion protein provided herein.

In one aspect, this disclosure provides a method of providing a MAD2019-H848A variant polypeptide to a cell, the method comprising: (a) obtaining a cell; and (b) providing the cell with a MAD2019-H848 variant polypeptide or a nucleic acid molecule encoding the MAD2019-H848A variant polypeptide.

In one aspect, this disclosure provides a method of providing a fusion protein to a cell, the method comprising: (a) obtaining a cell; and (b) providing the cell with a fusion protein provided herein, or a nucleic acid molecule encoding the fusion protein.

In one aspect, this disclosure provides a method of editing at least one eukaryotic cell, the method comprising: (a) introducing (i) a MAD2019-H848A variant polypeptide or a nucleic acid molecule encoding the MAD2019-H848A variant polypeptide to the at least one eukaryotic cell; and (ii) a guide RNA or a nucleic acid molecule encoding the guide RNA to the at least one eukaryotic cell, where the guide RNA comprises a nucleic acid sequence that is complementary to a target nucleic acid molecule within a genome of the eukaryotic cell; where the MAD2019-H848A variant polypeptide and the guide RNA form a nucleoprotein complex within the at least one eukaryotic cell, where the nucleoprotein complex cleaves one strand of the target nucleic acid molecule, and where at least one edit is made within the target nucleic acid molecule as compared to a control version of the target nucleic acid molecule; and (b) identifying at least one eukaryotic cell comprising the at least one edit.

In one aspect, this disclosure provides a method of editing at least one eukaryotic cell, the method comprising: (a) introducing (i) a fusion protein provided herein or a nucleic acid molecule encoding the fusion protein to the at least one eukaryotic cell; and (ii) a guide RNA or a nucleic acid molecule encoding the guide RNA to the at least one eukaryotic cell, where the guide RNA comprises a nucleic acid sequence that is complementary to a target nucleic acid molecule within a genome of the eukaryotic cell; where the fusion protein and the guide RNA form a nucleoprotein complex within the at least one eukaryotic cell, where the nucleoprotein complex cleaves one strand of the target nucleic acid molecule, and where at least one edit is made within the target nucleic acid molecule as compared to a control version of the target nucleic acid molecule; and (b) identifying at least one eukaryotic cell comprising the at least one edit.

In one aspect, this disclosure provides a guide RNA (gRNA) comprising a scaffold region having a nucleic acid sequence at least 80% identical to SEQ ID NO: 24. This disclosure also provides nucleoprotein complexes comprising a gRNA comprising a scaffold region having a nucleic acid sequence at least 85% identical to SEQ ID NO: 24.

### BRIEF DESCRIPTION OF THE SEQUENCES

Table 1 provides a list of nucleic acid sequences and amino acid sequences provided by this disclosure.

TABLE 1
Nucleic acid sequences and amino acid sequences.

| SEQ ID | | Sequence | |
|---|---|---|---|
| NO | Description | Type | Sequence |
| 1 | MAD2019-H848A | Amino | MTKPYSIGLDIGTNSVGWAVITDDYKVPSKKMK |
| | | acid | VLGNTSKKYIKKNLLGALLFDSGITAEGRRLKR |
| | | | TARRRYTRRRNRILYLQEIFSTEMATLDDAFFQ |
| | | | RLDDSFLVPDDKRDSKYPIFGNLVEEKAYHDEF |
| | | | PTIYHLRKYLADSTKKADLRLVYLALAHMIKYR |
| | | | GHFLIEGEFNSKNNDIQKNFQDELDTYNAIFES |
| | | | DLSLENSKQLEEIVKDKISKLEKKDRILKLEPG |
| | | | EKNSGIFSEFLKLIVGNQADEKKYENLDEKASL |
| | | | HFSKESYDEDLETLLGYIGDDYSDVELKAKKLY |
| | | | DAILLSGILTVTDNGTETPLSSAMIMRYKEHEE |
| | | | DLGLLKAYIRNISLKTYNEVENDDTKNGYAGYI |
| | | | DGKTNQEDFYVYLKKLLAKFEGADYFLEKIDRE |
| | | | DFLRKQRTFDNGSIPYQIHLQEMRAILDKQAKF |
| | | | YPFLAKNKERIEKILTFRIPYYVGPLARGNSDE |
| | | | AWSIRKRNEKITPWNFEDVIDKESSAEAFINRM |
| | | | TSFDLYLPEEKVLPKHSLLYETFTVYNELTKVR |
| | | | FIAEGMSDYQFLDSKQKKDIVRLYFKGKRKVKV |
| | | | TDKDIIEYLHAIDGYDGIELKGIEKQFNSSLST |
| | | | YHDLLNIINDKEFLDDSSNEAIIEEIIHTLTIF |
| | | | EDREMIKQRLSKFENIFDKSVLKKLSRRHYTGW |
| | | | GKLSAKLINGIRDEKSGNTILDYLIDDGISNRN |
| | | | FMQLIHDDALSFKKKIQKAQIIGDKDKDNIKEV |
| | | | VKSLPGSPAIKKGILQSIKIVDELVKVMGRKPE |
| | | | SIVVEMARENQYTNQGKSNSQQRLKRLEESLEE |
| | | | LGSKILKENIPAKLSKIDNNSLQNDRLYLYYLQ |
| | | | NGKDMYTGDDLDIDRLSNYDIDAIIPQAFLKDN |
| | | | SIDNKVLVSSASNRGKSDDVPSLEVVKKRKTLW |
| | | | YQLLKSKLISQRKEDNLTKAERGGLSPEDKAGE |
| | | | IQRQLVETRQITKHVARLLDEKENNKKDENNRA |
| | | | VRTVKIITLKSTLVSQFRKDFELYKVREINDFH |
| | | | HAHDAYLNAVVASALLKKYPKLEPEFVYGDYPK |
| | | | YNSFRERKSATEKVYFYSNIMNIFKKSISLADG |
| | | | RVIERPLIEVNEETGESVWNKESDLATVRRVLS |
| | | | YPQVNVVKKVEVQSGGFSKELVQPHGNSDKLIP |
| | | | RKTKKMIWDTKKYGGEDSPIVAYSVLVMAEREK |
| | | | GKSKKLKPVKELVRITIMEKESFKENTIDFLER |
| | | | RGLRNIQDENIILLPKESLFELENGRRRLLASA |
| | | | KELQKGNEFILPNKLVKLLYHAKNIHNTLEPEH |
| | | | LEYVESHRADFGKILDVVSVESEKYILAEAKLE |
| | | | KIKEIYRKNMNTEIHEMATAFINLLTFTSIGAP |
| | | | ATFKFFGHNIERKRYSSVAEILNATLIHQSVTG |
| | | | LYETRIDLGKLGED |
| 2 | Guide | Nucleic | GCCGCTACCCCGACCACATG |
| | | acid | |
| 3 | Guide | Nucleic | GCGGCAAACTGCCCGTGCCC |
| | | acid | |
| 4 | Homology arm | Nucleic | ACTGCACGCCGTGGCTCAGGGTGGTCACCAAAG |
| | | acid | TGGGCCATGGCACGGGCAGTTTG |
| 5 | Guide | Nucleic | GCCCGTGCCCTGGCCCACTT |
| | | acid | |
| 6 | Homology arm | Nucleic | ACTGCACGCCGTGGCTCAGGGTGGTCACTAGAG |
| | | acid | TGGGCCAGGGCAC |
| 7 | Guide | Nucleic | GCTGAAGCACTGCACGCCGT |
| | | acid | |
| 8 | Homology arm | Nucleic | CCACCCTGAGCCACGGCGTGCAGTGCTT |
| | | acid | |
| 9 | Guide | Nucleic | GGTGCTGCTTCATGTGGTCG |
| | | acid | |
| 10 | Homology arm | Nucleic | CCACCCTGAGCCACGGCGTGCAGTGCTTCAGCC |
| | | acid | GCTATCCTGACCACATGAAGCAG |
| 11 | Guide | Nucleic | GCCGCTACCCCGACCACATG |
| | | acid | |
| 12 | Tf1 (Schizo- | Amino | GSISSSKHTLSQMNKVSNIVKEPELPDIYKEFK |
| | saccharomyces | acid | DITADTNTEKLPKPIKGLEFEVELTQENYRLPI |
| | pombe) | | RNYPLPPGKMQAMNDEINQGLKSGIIRESKAIN |
| | | | ACPVMFVPKKEGTLRMVVDYKPLNKYVKPNIYP |
| | | | LPLIEQLLAKIQGSTIFTKLDLKSAYHLIRVRK |
| | | | GDEHKLAFRCPRGVFEYLVMPYGISTAPAHFQY |
| | | | FINTILGEAKESHVVCYMDDILIHSKSESEHVK |
| | | | HVKDVLQKLKNANLIINQAKCEFHQSQVKFIGY |
| | | | HISEKGFTPCQENIDKVLQWKQPKNRKELRQFL |
| | | | GSVNYLRKFIPKTSQLTHPLNKLLKKDVRWKWT |
| | | | PTQTQAIENIKQCLVSPPVLRHEDESKKILLET |
| | | | DASDVAVGAVLSQKHDDDKYYPVGYYSAKMSKA |
| | | | QLNYSVSDKEMLAIIKSLKHWRHYLESTIEPFK |
| | | | ILTDHRNLIGRITNESEPENKRLARWQLFLQDE |
| | | | NFEINYRPGSANHIADALSRIVDETEPIPKDSE |
| | | | DNSINFVNQISIGG |
| 13 | Tf1-D364N | Amino | GSISSSKHTLSQMNKVSNIVKEPELPDIYKEFK |
| | | acid | DITADTNTEKLPKPIKGLEFEVELTQENYRLPI |
| | | | RNYPLPPGKMQAMNDEINQGLKSGIIRESKAIN |
| | | | ACPVMFVPKKEGTLRMVVDYKPLNKYVKPNIYP |
| | | | LPLIEQLLAKIQGSTIFTKLDLKSAYHLIRVRK |
| | | | GDEHKLAFRCPRGVFEYLVMPYGISTAPAHFQY |
| | | | FINTILGEAKESHVVCYMDDILIHSKSESEHVK |
| | | | HVKDVLQKLKNANLIINQAKCEFHQSQVKFIGY |
| | | | HISEKGFTPCQENIDKVLQWKQPKNRKELRQFL |
| | | | GSVNYLRKFIPKTSQLTHPLNKLLKKDVRWKWT |
| | | | PTQTQAIENIKQCLVSPPVLRHFDESKKILLET |
| | | | NASDVAVGAVLSQKHDDDKYYPVGYYSAKMSKA |
| | | | QLNYSVSDKEMLAIIKSLKHWRHYLESTIEPFK |
| | | | ILTDHRNLIGRITNESEPENKRLARWQLFLQDE |
| | | | NFEINYRPGSANHIADALSRIVDETEPIPKDSE |
| | | | DNSINFVNQISIGG |
| 14 | Guide | Nucleic | GTTTAAGAGCTGGAAACAGCAAAGTTTAAATAA |
| | | acid | GGCTAGTCCGTATACAACGTGGAAACACGTGGC |
| | | | ACCGATTCGGTGC |
| 15 | Nuclear | Amino | MKRTADGSEFESPKKKRK |
| | localization | acid | |
| | signal | | |
| 16 | MAD2019 | Amino | VTKPYSIGLDIGTNSVGWAVITDDYKVPSKKMK |
| | nickase | acid | VLGNTSKKYIKKNLLGALLFDSGITAEGRRLKR |
| | | | TARRRYTRRRNRILYLQEIFSTEMATLDDAFFQ |
| | | | RLDDSFLVPDDKRDSKYPIFGNLVEEKAYHDEF |
| | | | PTIYHLRKYLADSTKKADLRLVYLALAHMIKYR |
| | | | GHFLIEGEFNSKNNDIQKNFQDELDTYNAIFES |
| | | | DLSLENSKQLEEIVKDKISKLEKKDRILKLFPG |
| | | | EKNSGIFSEFLKLIVGNQADFKKYENLDEKASL |
| | | | HESKESYDEDLETLLGYIGDDYSDVELKAKKLY |
| | | | DAILLSGILTVTDNGTETPLSSAMIMRYKEHEE |
| | | | DLGLLKAYIRNISLKTYNEVENDDTKNGYAGYI |
| | | | DGKTNQEDFYVYLKKLLAKFEGADYFLEKIDRE |
| | | | DFLRKQRTFDNGSIPYQIHLQEMRAILDKQAKF |
| | | | YPFLAKNKERIEKILTFRIPYYVGPLARGNSDE |
| | | | AWSIRKRNEKITPWNFEDVIDKESSAEAFINRM |
| | | | TSFDLYLPEEKVLPKHSLLYETFTVYNELTKVR |
| | | | FIAEGMSDYQFLDSKQKKDIVRLYFKGKRKVKV |
| | | | TDKDIIEYLHAIDGYDGIELKGIEKQENSSLST |
| | | | YHDLLNIINDKEFLDDSSNEAIIEEIIHTLTIF |
| | | | EDREMIKQRLSKFENIFDKSVLKKLSRRHYTGW |
| | | | GKLSAKLINGIRDEKSGNTILDYLIDDGISNRN |
| | | | FMQLIHDDALSFKKKIQKAQIIGDKDKDNIKEV |
| | | | VKSLPGSPAIKKGILQSIKIVDELVKVMGRKPE |
| | | | SIVVEMARENQYTNQGKSNSQQRLKRLEESLEE |
| | | | LGSKILKENIPAKLSKIDNNSLQNDRLYLYYLQ |
| | | | NGKDMYTGDDLDIDRLSNYDIDAIIPQAFLKDN |
| | | | SIDNKVLVSSASNRGKSDDVPSLEVVKKRKTLW |
| | | | YQLLKSKLISQRKEDNLTKAERGGLSPEDKAGE |
| | | | IQRQLVETRQITKHVARLLDEKENNKKDENNRA |
| | | | VRTVKIITLKSTLVSQFRKDFELYKVREINDFH |
| | | | HAHDAYLNAVVASALLKKYPKLEPEFVYGDYPK |
| | | | YNSFRERKSATEKVYFYSNIMNIFKKSISLADG |
| | | | RVIERPLIEVNEETGESVWNKESDLATVRRVLS |
| | | | YPQVNVVKKVEVQSGGFSKELVQPHGNSDKLIP |
| | | | RKTKKMIWDTKKYGGFDSPIVAYSVLVMAEREK |
| | | | GKSKKLKPVKELVRITIMEKESFKENTIDFLER |
| | | | RGLRNIQDENIILLPKFSLFELENGRRRLLASA |
| | | | KELQKGNEFILPNKLVKLLYHAKNIHNTLEPEH |
| | | | LEYVESHRADFGKILDVVSVESEKYILAEAKLE |
| | | | KIKEIYRKNMNTEIHEMATAFINLLTFTSIGAP |
| | | | ATFKFFGHNIERKRYSSVAEILNATLIHQSVTG |
| | | | LYETRIDLGKLGED |
| 17 | Linker | Amino | NSGGSSGGSSGSETPGTSESATPESSGGSSGGS |
| | | acid | S |
| 18 | MLV reverse | Amino | TLNIEDEYRLHETSKEPDVSLGSTWLSDFPQAW |
| | transcriptase | acid | AETGGMGLAVRQAPLIIPLKATSTPVSIKQYPM |
| | | | SQEARLGIKPHIQRLLDQGILVPCQSPWNTPLL |
| | | | PVKKPGTNDYRPVQDLREVNKRVEDIHPTVPNP |
| | | | YNLLSGLPPSHQWYTVLDLKDAFFCLRLHPTSQ |
| | | | PLFAFEWRDPEMGISGQLTWTRLPQGFKNSPTL |
| | | | FNEALHRDLADFRIQHPDLILLQYVDDLLLAAT |
| | | | SELDCQQGTRALLQTLGNLGYRASAKKAQICQK |
| | | | QVKYLGYLLKEGQRWLTEARKETVMGQPTPKTP |
| | | | RQLREFLGKAGFCRLFIPGFAEMAAPLYPLTKP |
| | | | GTLFNWGPDQQKAYQEIKQALLTAPALGLPDLT |
| | | | KPFELFVDEKQGYAKGVLTQKLGPWRRPVAYLS |
| | | | KKLDPVAAGWPPCLRMVAAIAVLTKDAGKLTMG |
| | | | QPLVILAPHAVEALVKQPPDRWLSNARMTHYQA |
| | | | LLLDTDRVQFGPVVALNPATLLPLPEEGLQHNC |
| | | | LDILAEAHGTRPDLTDQPLPDADHTWYTDGSSL |
| | | | LQEGQRKAGAAVTTETEVIWAKALPAGTSAQRA |
| | | | ELIALTQALKMAEGKKLNVYTDSRYAFATAHIH |
| | | | GEIYRRRGWLTSEGKEIKNKDEILALLKALFLP |
| | | | KRLSIIHCPGHQKGHSAEARGNRMADQAARKAA |
| | | | ITETPDTSTLLIENSSP |
| 19 | Nuclear | Amino | SKRTADGSEFEPKKKRKV |
| | localization | acid | |
| | signal | | |
| 20 | CREATE fusion | Amino | MKRTADGSEFESPKKKRKVVTKPYSIGLDIGTN |
| | enzyme 19 | acid | SVGWAVITDDYKVPSKKMKVLGNTSKKYIKKNL |
| | (CFE19) | | LGALLFDSGITAEGRRLKRTARRRYTRRRNRIL |
| | | | YLQEIFSTEMATLDDAFFQRLDDSELVPDDKRD |
| | | | SKYPIFGNLVEEKAYHDEFPTIYHLRKYLADST |
| | | | KKADLRLVYLALAHMIKYRGHFLIEGEENSKNN |
| | | | DIQKNFQDFLDTYNAIFESDLSLENSKQLEEIV |
| | | | KDKISKLEKKDRILKLFPGEKNSGIFSEFLKLI |
| | | | VGNQADFKKYFNLDEKASLHESKESYDEDLETL |
| | | | LGYIGDDYSDVELKAKKLYDAILLSGILTVTDN |
| | | | GTETPLSSAMIMRYKEHEEDLGLLKAYIRNISL |
| | | | KTYNEVFNDDTKNGYAGYIDGKTNQEDFYVYLK |
| | | | KLLAKFEGADYFLEKIDREDELRKQRTEDNGSI |
| | | | PYQIHLQEMRAILDKQAKFYPFLAKNKERIEKI |
| | | | LTFRIPYYVGPLARGNSDFAWSIRKRNEKITPW |
| | | | NEEDVIDKESSAEAFINRMTSEDLYLPEEKVLP |
| | | | KHSLLYETFTVYNELTKVRFIAEGMSDYQFLDS |
| | | | KQKKDIVRLYFKGKRKVKVTDKDIIEYLHAIDG |
| | | | YDGIELKGIEKQFNSSLSTYHDLLNIINDKEFL |
| | | | DDSSNEAIIEEIIHTLTIFEDREMIKQRLSKFE |
| | | | NIFDKSVLKKLSRRHYTGWGKLSAKLINGIRDE |
| | | | KSGNTILDYLIDDGISNRNEMQLIHDDALSFKK |
| | | | KIQKAQIIGDKDKDNIKEVVKSLPGSPAIKKGI |
| | | | LQSIKIVDELVKVMGRKPESIVVEMARENQYTN |
| | | | QGKSNSQQRLKRLEESLEELGSKILKENIPAKL |
| | | | SKIDNNSLQNDRLYLYYLQNGKDMYTGDDLDID |
| | | | RLSNYDIDAIIPQAFLKDNSIDNKVLVSSASNR |
| | | | GKSDDVPSLEVVKKRKTLWYQLLKSKLISQRKE |
| | | | DNLTKAERGGLSPEDKAGFIQRQLVETRQITKH |
| | | | VARLLDEKENNKKDENNRAVRTVKIITLKSTLV |
| | | | SQFRKDFELYKVREINDFHHAHDAYLNAVVASA |
| | | | LLKKYPKLEPEFVYGDYPKYNSFRERKSATEKV |
| | | | YFYSNIMNIFKKSISLADGRVIERPLIEVNEET |
| | | | GESVWNKESDLATVRRVLSYPQVNVVKKVEVQS |
| | | | GGFSKELVQPHGNSDKLIPRKTKKMIWDTKKYG |
| | | | GFDSPIVAYSVLVMAEREKGKSKKLKPVKELVR |
| | | | ITIMEKESFKENTIDFLERRGLRNIQDENIILL |
| | | | PKFSLFELENGRRRLLASAKELQKGNEFILPNK |
| | | | LVKLLYHAKNIHNTLEPEHLEYVESHRADEGKI |
| | | | LDVVSVESEKYILAEAKLEKIKEIYRKNMNTEI |
| | | | HEMATAFINLLTFTSIGAPATFKFFGHNIERKR |
| | | | YSSVAEILNATLIHQSVTGLYETRIDLGKLGED |
| | | | NSGGSSGGSSGSETPGTSESATPESSGGSSGGS |
| | | | STLNIEDEYRLHETSKEPDVSLGSTWLSDEPQA |
| | | | WAETGGMGLAVRQAPLIIPLKATSTPVSIKQYP |
| | | | MSQEARLGIKPHIQRLLDQGILVPCQSPWNTPL |
| | | | LPVKKPGTNDYRPVQDLREVNKRVEDIHPTVPN |
| | | | PYNLLSGLPPSHQWYTVLDLKDAFFCLRLHPTS |
| | | | QPLFAFEWRDPEMGISGQLTWTRLPQGEKNSPT |
| | | | LFNEALHRDLADFRIQHPDLILLQYVDDLLLAA |
| | | | TSELDCQQGTRALLQTLGNLGYRASAKKAQICQ |
| | | | KQVKYLGYLLKEGQRWLTEARKETVMGQPTPKT |
| | | | PRQLREFLGKAGFCRLFIPGFAEMAAPLYPLTK |
| | | | PGTLFNWGPDQQKAYQEIKQALLTAPALGLPDL |
| | | | TKPFELFVDEKQGYAKGVLTQKLGPWRRPVAYL |
| | | | SKKLDPVAAGWPPCLRMVAAIAVLTKDAGKLTM |
| | | | GQPLVILAPHAVEALVKQPPDRWLSNARMTHYQ |
| | | | ALLLDTDRVQFGPVVALNPATLLPLPEEGLQHN |
| | | | CLDILAEAHGTRPDLTDQPLPDADHTWYTDGSS |
| | | | LLQEGQRKAGAAVTTETEVIWAKALPAGTSAQR |
| | | | AELIALTQALKMAEGKKLNVYTDSRYAFATAHI |
| | | | HGEIYRRRGWLTSEGKEIKNKDEILALLKALFL |
| | | | PKRLSIIHCPGHQKGHSAEARGNRMADQAARKA |
| | | | AITETPDTSTLLIENSSPSGGSKRTADGSEFEP |
| | | | KKKRKV |
| 21 | CFE19 variant | Amino | MKRTADGSEFESPKKKRKVVTKPYSIGLDIGTN |
| | with V1143T | acid | SVGWAVITDDYKVPSKKMKVLGNTSKKYIKKNL |
| | substitution | | LGALLFDSGITAEGRRLKRTARRRYTRRRNRIL |
| | | | YLQEIFSTEMATLDDAFFQRLDDSELVPDDKRD |
| | | | SKYPIFGNLVEEKAYHDEFPTIYHLRKYLADST |
| | | | KKADLRLVYLALAHMIKYRGHFLIEGEENSKNN |
| | | | DIQKNFQDELDTYNAIFESDLSLENSKQLEEIV |
| | | | KDKISKLEKKDRILKLFPGEKNSGIFSEFLKLI |
| | | | VGNQADFKKYFNLDEKASLHESKESYDEDLETL |
| | | | LGYIGDDYSDVELKAKKLYDAILLSGILTVTDN |
| | | | GTETPLSSAMIMRYKEHEEDLGLLKAYIRNISL |
| | | | KTYNEVENDDTKNGYAGYIDGKTNQEDFYVYLK |
| | | | KLLAKFEGADYFLEKIDREDELRKQRTEDNGSI |
| | | | PYQIHLQEMRAILDKQAKFYPFLAKNKERIEKI |
| | | | LTFRIPYYVGPLARGNSDFAWSIRKRNEKITPW |
| | | | NEEDVIDKESSAEAFINRMTSEDLYLPEEKVLP |
| | | | KHSLLYETFTVYNELTKVRFIAEGMSDYQELDS |
| | | | KQKKDIVRLYFKGKRKVKVTDKDIIEYLHAIDG |
| | | | YDGIELKGIEKQFNSSLSTYHDLLNIINDKEFL |
| | | | DDSSNEAIIEEIIHTLTIFEDREMIKQRLSKFE |
| | | | NIFDKSVLKKLSRRHYTGWGKLSAKLINGIRDE |
| | | | KSGNTILDYLIDDGISNRNEMQLIHDDALSFKK |
| | | | KIQKAQIIGDKDKDNIKEVVKSLPGSPAIKKGI |
| | | | LQSIKIVDELVKVMGRKPESIVVEMARENQYTN |
| | | | QGKSNSQQRLKRLEESLEELGSKILKENIPAKL |
| | | | SKIDNNSLQNDRLYLYYLQNGKDMYTGDDLDID |
| | | | RLSNYDIDAIIPQAFLKDNSIDNKVLVSSASNR |
| | | | GKSDDVPSLEVVKKRKTLWYQLLKSKLISQRKF |
| | | | DNLTKAERGGLSPEDKAGFIQRQLVETRQITKH |
| | | | VARLLDEKENNKKDENNRAVRTVKIITLKSTLV |
| | | | SQFRKDFELYKVREINDFHHAHDAYLNAVVASA |
| | | | LLKKYPKLEPEFVYGDYPKYNSFRERKSATEKV |
| | | | YFYSNIMNIFKKSISLADGRVIERPLIEVNEET |
| | | | GESVWNKESDLATVRRVLSYPQVNVVKKVEVQS |
| | | | GGFSKELVQPHGNSDKLIPRKTKKMIWDTKKYG |
| | | | GFDSPITAYSVLVMAEREKGKSKKLKPVKELVR |
| | | | ITIMEKESFKENTIDFLERRGLRNIQDENIILL |
| | | | PKFSLFELENGRRRLLASAKELQKGNEFILPNK |
| | | | LVKLLYHAKNIHNTLEPEHLEYVESHRADEGKI |
| | | | LDVVSVESEKYILAEAKLEKIKEIYRKNMNTEI |
| | | | HEMATAFINLLTFTSIGAPATFKFFGHNIERKR |
| | | | YSSVAEILNATLIHQSVTGLYETRIDLGKLGED |
| | | | NSGGSSGGSSGSETPGTSESATPESSGGSSGGS |
| | | | STLNIEDEYRLHETSKEPDVSLGSTWLSDFPQA |
| | | | WAETGGMGLAVRQAPLIIPLKATSTPVSIKQYP |
| | | | MSQEARLGIKPHIQRLLDQGILVPCQSPWNTPL |
| | | | LPVKKPGTNDYRPVQDLREVNKRVEDIHPTVPN |
| | | | PYNLLSGLPPSHQWYTVLDLKDAFFCLRLHPTS |
| | | | QPLFAFEWRDPEMGISGQLTWTRLPQGFKNSPT |
| | | | LFNEALHRDLADFRIQHPDLILLQYVDDLLLAA |
| | | | TSELDCQQGTRALLQTLGNLGYRASAKKAQICQ |
| | | | KQVKYLGYLLKEGQRWLTEARKETVMGQPTPKT |
| | | | PRQLREFLGKAGFCRLFIPGFAEMAAPLYPLTK |
| | | | PGTLFNWGPDQQKAYQEIKQALLTAPALGLPDL |
| | | | TKPFELFVDEKQGYAKGVLTQKLGPWRRPVAYL |
| | | | SKKLDPVAAGWPPCLRMVAAIAVLTKDAGKLTM |
| | | | GQPLVILAPHAVEALVKQPPDRWLSNARMTHYQ |
| | | | ALLLDTDRVQFGPVVALNPATLLPLPEEGLQHN |
| | | | CLDILAEAHGTRPDLTDQPLPDADHTWYTDGSS |
| | | | LLQEGQRKAGAAVTTETEVIWAKALPAGTSAQR |
| | | | AELIALTQALKMAEGKKLNVYTDSRYAFATAHI |
| | | | HGEIYRRRGWLTSEGKEIKNKDEILALLKALFL |
| | | | PKRLSIIHCPGHQKGHSAEARGNRMADQAARKA |
| | | | AITETPDTSTLLIENSSPSGGSKRTADGSEFEP |
| | | | KKKRKV |
| 22 | CFE19 variant | Amino | MKRTADGSEFESPKKKRKVVTKPYSIGLDIGTN |
| | with V1143T | acid | SVGWAVITDDYKVPSKKMKVLGNTSKKYIKKNL |
| | substitution | | LGALLEDSGITAEGRRLKRTARRRYTRRRNRIL |
| | and | | YLQEIFSTEMATLDDAFFQRLDDSELVPDDKRD |
| | L500R, D700A, | | SKYPIFGNLVEEKAYHDEFPTIYHLRKYLADST |
| | D701P, K720S, | | KKADLRLVYLALAHMIKYRGHFLIEGEENSKNN |
| | and I1142K | | DIQKNFQDFLDTYNAIFESDLSLENSKQLEEIV |
| | substitutions | | KDKISKLEKKDRILKLFPGEKNSGIFSEFLKLI |
| | | | VGNQADFKKYENLDEKASLHESKESYDEDLETL |
| | | | LGYIGDDYSDVELKAKKLYDAILLSGILTVTDN |
| | | | GTETPLSSAMIMRYKEHEEDLGLLKAYIRNISL |
| | | | KTYNEVENDDTKNGYAGYIDGKTNQEDFYVYLK |
| | | | KLLAKFEGADYFLEKIDREDELRKQRTEDNGSI |
| | | | PYQIHLQEMRAILDKQAKFYPFLAKNKERIEKI |
| | | | LTFRIPYYVGPLARGNSDFAWSIRKRNEKITPW |
| | | | NEEDVIDKESSAEAFINRMTSEDRYLPEEKVLP |
| | | | KHSLLYETFTVYNELTKVRFIAEGMSDYQELDS |
| | | | KQKKDIVRLYFKGKRKVKVTDKDIIEYLHAIDG |
| | | | YDGIELKGIEKQFNSSLSTYHDLLNIINDKEFL |
| | | | DDSSNEAIIEEIIHTLTIFEDREMIKQRLSKFE |
| | | | NIFDKSVLKKLSRRHYTGWGKLSAKLINGIRDE |
| | | | KSGNTILDYLIDDGISNRNFMQLIHAPALSEKK |
| | | | KIQKAQIIGDKDSDNIKEVVKSLPGSPAIKKGI |
| | | | LQSIKIVDELVKVMGRKPESIVVEMARENQYTN |
| | | | QGKSNSQQRLKRLEESLEELGSKILKENIPAKL |
| | | | SKIDNNSLQNDRLYLYYLQNGKDMYTGDDLDID |
| | | | RLSNYDIDAIIPQAFLKDNSIDNKVLVSSASNR |
| | | | GKSDDVPSLEVVKKRKTLWYQLLKSKLISQRKF |
| | | | DNLTKAERGGLSPEDKAGFIQRQLVETRQITKH |
| | | | VARLLDEKENNKKDENNRAVRTVKIITLKSTLV |
| | | | SQFRKDFELYKVREINDFHHAHDAYLNAVVASA |
| | | | LLKKYPKLEPEFVYGDYPKYNSFRERKSATEKV |
| | | | YFYSNIMNIFKKSISLADGRVIERPLIEVNEET |
| | | | GESVWNKESDLATVRRVLSYPQVNVVKKVEVQS |
| | | | GGFSKELVQPHGNSDKLIPRKTKKMIWDTKKYG |
| | | | GFDSPKTAYSVLVMAEREKGKSKKLKPVKELVR |
| | | | ITIMEKESFKENTIDFLERRGLRNIQDENIILL |
| | | | PKFSLFELENGRRRLLASAKELQKGNEFILPNK |
| | | | LVKLLYHAKNIHNTLEPEHLEYVESHRADFGKI |
| | | | LDVVSVESEKYILAEAKLEKIKEIYRKNMNTEI |
| | | | HEMATAFINLLTFTSIGAPATEKFFGHNIERKR |
| | | | YSSVAEILNATLIHQSVTGLYETRIDLGKLGED |
| | | | NSGGSSGGSSGSETPGTSESATPESSGGSSGGS |
| | | | STLNIEDEYRLHETSKEPDVSLGSTWLSDEPQA |
| | | | WAETGGMGLAVRQAPLIIPLKATSTPVSIKQYP |
| | | | MSQEARLGIKPHIQRLLDQGILVPCQSPWNTPL |
| | | | LPVKKPGTNDYRPVQDLREVNKRVEDIHPTVPN |
| | | | PYNLLSGLPPSHQWYTVLDLKDAFFCLRLHPTS |
| | | | QPLFAFEWRDPEMGISGQLTWTRLPQGEKNSPT |
| | | | LFNEALHRDLADFRIQHPDLILLQYVDDLLLAA |
| | | | TSELDCQQGTRALLQTLGNLGYRASAKKAQICQ |
| | | | KQVKYLGYLLKEGQRWLTEARKETVMGQPTPKT |
| | | | PRQLREFLGKAGFCRLFIPGFAEMAAPLYPLTK |
| | | | PGTLFNWGPDQQKAYQEIKQALLTAPALGLPDL |
| | | | TKPFELFVDEKQGYAKGVLTQKLGPWRRPVAYL |
| | | | SKKLDPVAAGWPPCLRMVAAIAVLTKDAGKLTM |
| | | | GQPLVILAPHAVEALVKQPPDRWLSNARMTHYQ |
| | | | ALLLDTDRVQFGPVVALNPATLLPLPEEGLQHN |
| | | | CLDILAEAHGTRPDLTDQPLPDADHTWYTDGSS |
| | | | LLQEGQRKAGAAVTTETEVIWAKALPAGTSAQR |
| | | | AELIALTQALKMAEGKKLNVYTDSRYAFATAHI |
| | | | HGEIYRRRGWLTSEGKEIKNKDEILALLKALFL |
| | | | PKRLSIIHCPGHQKGHSAEARGNRMADQAARKA |
| | | | AITETPDTSTLLIENSSPSGGSKRTADGSEFEP |
| | | | KKKRKV |
| 23 | CFE19 variant | Amino | MKRTADGSEFESPKKKRKVVTKPYSIGLDIGTN |
| | with V1143T | acid | SVGWAVITDDYKVPSKKMKVLGNTSKKYIKKNL |
| | substitution | | LGALLFDSGITAEGRRLKRTARRRYTRRRNRIL |
| | and | | YLQEIFSTEMATLDDAFFQRLDDSFLVPDDKRD |
| | L500R, D700A, | | SKYPIFGNLVEEKAYHDEFPTIYHLRKYLADST |
| | D701P, and | | KKADLRLVYLALAHMIKYRGHFLIEGEFNSKNN |
| | K720S | | DIQKNFQDELDTYNAIFESDLSLENSKQLEEIV |
| | substitutions | | KDKISKLEKKDRILKLFPGEKNSGIFSEFLKLI |
| | | | VGNQADEKKYFNLDEKASLHFSKESYDEDLETL |
| | | | LGYIGDDYSDVELKAKKLYDAILLSGILTVTDN |
| | | | GTETPLSSAMIMRYKEHEEDLGLLKAYIRNISL |
| | | | KTYNEVENDDTKNGYAGYIDGKTNQEDFYVYLK |
| | | | KLLAKFEGADYFLEKIDREDELRKQRTEDNGSI |
| | | | PYQIHLQEMRAILDKQAKFYPFLAKNKERIEKI |
| | | | LTFRIPYYVGPLARGNSDFAWSIRKRNEKITPW |
| | | | NEEDVIDKESSAEAFINRMTSEDRYLPEEKVLP |
| | | | KHSLLYETFTVYNELTKVRFIAEGMSDYQFLDS |
| | | | KQKKDIVRLYFKGKRKVKVTDKDIIEYLHAIDG |
| | | | YDGIELKGIEKQFNSSLSTYHDLLNIINDKEFL |
| | | | DDSSNEAIIEEIIHTLTIFEDREMIKQRLSKFE |
| | | | NIFDKSVLKKLSRRHYTGWGKLSAKLINGIRDE |
| | | | KSGNTILDYLIDDGISNRNEMQLIHAPALSEKK |
| | | | KIQKAQIIGDKDSDNIKEVVKSLPGSPAIKKGI |
| | | | LQSIKIVDELVKVMGRKPESIVVEMARENQYTN |
| | | | QGKSNSQQRLKRLEESLEELGSKILKENIPAKL |
| | | | SKIDNNSLQNDRLYLYYLQNGKDMYTGDDLDID |
| | | | RLSNYDIDAIIPQAFLKDNSIDNKVLVSSASNR |
| | | | GKSDDVPSLEVVKKRKTLWYQLLKSKLISQRKE |
| | | | DNLTKAERGGLSPEDKAGFIQRQLVETRQITKH |
| | | | VARLLDEKENNKKDENNRAVRTVKIITLKSTLV |
| | | | SQFRKDFELYKVREINDFHHAHDAYLNAVVASA |
| | | | LLKKYPKLEPEFVYGDYPKYNSFRERKSATEKV |
| | | | YFYSNIMNIFKKSISLADGRVIERPLIEVNEET |
| | | | GESVWNKESDLATVRRVLSYPQVNVVKKVEVQS |
| | | | GGFSKELVQPHGNSDKLIPRKTKKMIWDTKKYG |
| | | | GFDSPITAYSVLVMAEREKGKSKKLKPVKELVR |
| | | | ITIMEKESFKENTIDFLERRGLRNIQDENIILL |
| | | | PKFSLFELENGRRRLLASAKELQKGNEFILPNK |
| | | | LVKLLYHAKNIHNTLEPEHLEYVESHRADEGKI |
| | | | LDVVSVESEKYILAEAKLEKIKEIYRKNMNTEI |
| | | | HEMATAFINLLTFTSIGAPATEKFFGHNIERKR |
| | | | YSSVAEILNATLIHQSVTGLYETRIDLGKLGED |
| | | | NSGGSSGGSSGSETPGTSESATPESSGGSSGGS |
| | | | STLNIEDEYRLHETSKEPDVSLGSTWLSDFPQA |
| | | | WAETGGMGLAVRQAPLIIPLKATSTPVSIKQYP |
| | | | MSQEARLGIKPHIQRLLDQGILVPCQSPWNTPL |
| | | | LPVKKPGTNDYRPVQDLREVNKRVEDIHPTVPN |
| | | | PYNLLSGLPPSHQWYTVLDLKDAFFCLRLHPTS |
| | | | QPLFAFEWRDPEMGISGQLTWTRLPQGFKNSPT |
| | | | LFNEALHRDLADFRIQHPDLILLQYVDDLLLAA |
| | | | TSELDCQQGTRALLQTLGNLGYRASAKKAQICQ |
| | | | KQVKYLGYLLKEGQRWLTEARKETVMGQPTPKT |
| | | | PRQLREFLGKAGFCRLFIPGFAEMAAPLYPLTK |
| | | | PGTLFNWGPDQQKAYQEIKQALLTAPALGLPDL |
| | | | TKPFELFVDEKQGYAKGVLTQKLGPWRRPVAYL |
| | | | SKKLDPVAAGWPPCLRMVAAIAVLTKDAGKLTM |
| | | | GQPLVILAPHAVEALVKQPPDRWLSNARMTHYQ |
| | | | ALLLDTDRVQFGPVVALNPATLLPLPEEGLQHN |
| | | | CLDILAEAHGTRPDLTDQPLPDADHTWYTDGSS |
| | | | LLQEGQRKAGAAVTTETEVIWAKALPAGTSAQR |
| | | | AELIALTQALKMAEGKKLNVYTDSRYAFATAHI |
| | | | HGEIYRRRGWLTSEGKEIKNKDEILALLKALFL |
| | | | PKRLSIIHCPGHQKGHSAEARGNRMADQAARKA |
| | | | AITETPDTSTLLIENSSPSGGSKRTADGSEFEP |
| | | | KKKRKV |
| 24 | gRNA scaffold | Nucleic | GTTTAAGAGCTGGAAACAGCAAAGTTTAAATAA |
| | region | acid | GGCTAGTCCGTATACAACGTGGAAACACGTGGC |
| | | | ACCGATTCGGTGC |
| 25 | Gquad | Nucleic | ACTAACGGTGGTGGTGG |
| | stabilizing | acid | |
| | sequence | | |
| | element | | |
| 26 | Guide | Nucleic | GCTTCATGTGGTCGGGGTAG |
| | | acid | |
| 27 | Homology arm | Nucleic | CCACCCTGAGCCACGGCGTGCAGTGCTTCAGTC |
| | | acid | GCTACCCCGACCACATG |
| 28 | Guide | Nucleic | GGCTGAAGCACTGCACGCCG |
| | | acid | |
| 29 | Homology arm | Nucleic | TGACCACCCTGTCGCATGGCGTGCAGTGCTTC |
| | | acid | |

### BRIEF DESCRIPTION OF THE DRAWINGS

Without being limited by any scientific theory, FIG. 1 depicts a mechanism for CREATE fusion editing.

FIG. 2 depicts an example of a workflow for screening nickases for cutting activity and CREATE fusion activity.

FIG. 3A depicts the results of editing with the nickase fusion enzyme MAD2019-H848A::reverse transcriptase in HEK293T cells. FIG. 3B depicts the results of editing with the nickase fusion enzyme MAD2019-H848A::reverse transcriptase in induced pluripotent stem cells.

FIG. 4 depicts the results of GREEN FLUORSECENCE PROTEIN (GFP) to BLUE FLUORESCENCE PROTEIN (BFP) editing with MAD2019-H848A (SEQ ID NO: 1) fused to the reverse transcriptase Tf1 (SEQ ID NO: 12) or MAD2019-H848A fused to the reverse transcriptase Tf1-D364N (SEQ ID NO: 13).

FIG. 5 depicts the results of GFP to BFP editing with CFE19 (SEQ ID NO: 20) and CFE19 variants (e.g., SEQ ID NOs: 21 to 23 et al.).

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6^{th} edition, 2002, McGraw-Hill, New York), or the “Oxford Dictionary of Biology” (6^{th} edition, 2008, Oxford University Press, Oxford and New York).

The practice of the techniques described herein may employ, unless otherwise indicated, conventional techniques and descriptions of organic chemistry, polymer technology, molecular biology (including recombinant techniques), cell biology, biochemistry and sequencing technology, which are within the skill of those who practice in the art. Such conventional techniques include polymer array synthesis and hybridization and ligation of polynucleotides. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3^{rd} Ed., W. H. Freeman Pub., New York, N.Y.; Viral Vectors (Kaplift & Loewy, eds., Academic Press 1995); all of which are herein incorporated in their entirety by reference for all purposes. For mammalian/stem cell culture and methods see, e.g., Basic Cell Culture Protocols, Fourth Ed. (Helgason & Miller, eds., Humana Press 2005); Culture of Animal Cells, Seventh Ed. (Freshney, ed., Humana Press 2016); Microfluidic Cell Culture, Second Ed. (Borenstein, Vandon, Tao & Charest, eds., Elsevier Press 2018); Human Cell Culture (Hughes, ed., Humana Press 2011); 3D Cell Culture (Koledova, ed., Humana Press 2017); Cell and Tissue Culture: Laboratory Procedures in Biotechnology (Doyle & Griffiths, eds., John Wiley & Sons 1998); Essential Stem Cell Methods, (Lanza & Klimanskaya, eds., Academic Press 2011); Stem Cell Therapies: Opportunities for Ensuring the Quality and Safety of Clinical Offerings: Summary of a Joint Workshop (Board on Health Sciences Policy, National Academies Press 2014); Essentials of Stem Cell Biology, Third Ed., (Lanza & Atala, eds., Academic Press 2013); and Handbook of Stem Cells, (Atala & Lanza, eds., Academic Press 2012). CRISPR-specific techniques can be found in, e.g., Genome Editing and Engineering from TALENs and CRISPRs to Molecular Surgery, Appasani and Church (2018); and CRISPR: Methods and Protocols, Lindgren and Charpentier (2015).

Any references cited herein, including, e.g., all patents, published patent applications, and non-patent publications, are incorporated herein by reference in their entireties.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc.

The term “and/or” when used in a list of two or more items means any one of the listed items by itself or in combination with any one or more of the other listed items. For example, the expression “A and/or B” is intended to mean either or both of A and B—e.g., A alone, B alone, or A and B in combination. The expression “A, B and/or C” is intended to mean A alone, B alone, C alone, A and B in combination, A and C in combination, B and C in combination, or A, B, and C in combination.

When a range of numbers is provided herein, the range is understood to inclusive of the edges of the range as well as any number between the defined edges of the range. For example, “between 1 and 10” includes any number between 1 and 10, as well as the number 1 and the number 10.

As used herein, the singular form “a,” “an,” and “the” include plural references unless the context clearly dictates otherwise. For example, the term “a compound” or “at least one compound” may include a plurality of compounds, including mixtures thereof.

The nucleic acid guided nickases provided herein are employed to allow one to perform nucleic acid nickase fusion-directed genome editing to introduce desired edits to a live eukaryotic cell. The nucleic acid guided nickases provided herein are also employed to allow one to perform nucleic acid nickase fusion-directed genome editing to introduce desired edits to a target nucleic acid molecule in an in vitro setting.

As used herein, a “nickase” refers to a nuclease that cleaves a single-strand of double-stranded DNA molecule (e.g., a nickase “nicks” the DNA molecule). Nickases do not cleave both strands of a double-stranded DNA molecule. Examples of nickases or nucleic acid-guided nucleases can be found in U.S. Pat. Nos. 9,982,279; 10,337,028; 10,435,714; 10,011,849; 10,626,416; 10,604,746; 10,665,114; 10,640,754; 10,689,669; 10,876,102; 10,883,077; 10,704,033; 10,745,678; 10,724,021; 10,767,169; 10,870,761; 10,883,077; 11,053,485; 11,085,030; 11,200,089; 11,268,078; 11,293,115; 11,306,298; and 11,332,742; and U.S. Patent Application Publication No. 2021/0214671.

Nickases can be derived or engineered from nucleases that cleave both strands of a double-stranded DNA molecule. In some instances, nickases are derived from CRISPR-Cas enzymes. A non-limiting example of an engineered nickase is MAD2019 and variants, including MAD2019-H848A (SEQ ID NO: 1) and the variants provided in Table 2. In an aspect, a nickase can be guided by a nucleic acid molecule (e.g. a guide) to a specific site within a target nucleic acid molecule. Nickases that are guided by nucleic acid molecules are referred to as “nucleic acid-guided nickases.”

Nucleic acid-guided nickases provided herein can be combined with a reverse transcriptase to generate a fused enzyme (e.g., a fusion protein) that both binds and nicks a target nucleic acid molecule in a sequence-specific manner and is capable of utilizing a repair template (e.g., a homology arm) to incorporate nucleotides into the target nucleic acid sequence at the site of the nick. Such enzymes can be referred to as “nucleic acid-guided nickase fusion enzymes,” “CREATE fusion enzymes,” or “CF enzymes” herein. The process of using such enzymes to edit a target nucleic acid molecule is referred to as “CREATE fusion editing.”

FIG. 1 provides a simplified graphic of the process of CREATE fusion editing, including the steps of editing, flap equilibration, flap excision and repair, and DNA replication and cell division.

In addition to nucleic acid-guided nickases, various guide RNA scaffold sequences and guide RNA (gRNA) enhancements have been identified to be used with the nickases and fusion proteins provided herein to improve editing efficiency. See, for example, U.S. Pat. No. 11,268,078 and U.S. patent application Ser. No. 17/538,066, filed Nov. 30, 2021.

Without being limited by any scientific theory, a nucleic acid-guided nickase fusion enzyme complexed with a guide nucleic acid in a cell can nick the genome of the cell within a target nucleic acid molecule. The guide nucleic acid assists the nucleic acid-guided nickase fusion enzyme with recognizing and cutting one strand of the target nucleic acid molecule. By manipulating the nucleotide sequence of the guide nucleic acid molecule, the nucleic acid-guided nickase fusion enzyme can be programmed to target any DNA sequence for cleavage as long as an appropriate protospacer adjacent motif (PAM) is positioned nearby. As is known in the art, the precise sequence and length requirements for a PAM varies between different nucleic acid-guided nucleases and nucleic acid-guided nickases. However, PAMs typically comprise between 2 nucleotides and 10 nucleotides in length (most typically between 2 nucleotides and 6 nucleotides), and they are usually adjacent to, or within 10 nucleotides of a desired nick site. A non-limiting example of a PAM site is the sequence 5′-NGG-3′. A PAM can be positioned 5′ or 3′ of a desired nick site within a target nucleic acid molecule.

In an aspect, an edit comprises an edit to a PAM. In an aspect, an edit to a PAM results in the removal of the PAM from a target nucleic acid molecule. In an aspect, an edit to a PAM results in the inactivation of the PAM in a target nucleic acid molecule.

The nickase MAD2019-H848A was modified using CREATE fusion editing to identify “MAD2019-H848A variant polypeptides.” MAD2019-H848A variant polypeptides comprise at least one amino acid change as compared to SEQ ID NO: 1, but they also maintain an alanine at position 848 according to the numbering of SEQ ID NO: 1. In an aspect, a MAD2019-H848A variant polypeptide cleaves one strand (e.g., nicks) of a double-stranded DNA molecule. In an aspect, a MAD2019-H848A variant polypeptide does not cleave both strands of a double-stranded DNA molecule. Although not intended to be limiting, specific examples of MAD2019-H848A variant polypeptides are provided in Table 2.

In an aspect, this disclosure provides a MAD2019-H848A variant polypeptide comprising an amino acid sequence at least 70% identical or similar to SEQ ID NO: 1, where the MAD2019-H848A variant polypeptide comprises an alanine at position 848 according to SEQ ID NO: 1. In an aspect, this disclosure provides a MAD2019-H848A variant polypeptide comprising an amino acid sequence at least 75% identical or similar to SEQ ID NO: 1, where the MAD2019-H848A variant polypeptide comprises an alanine at position 848 according to SEQ ID NO: 1. In an aspect, this disclosure provides a MAD2019-H848A variant polypeptide comprising an amino acid sequence at least 80% identical or similar to SEQ ID NO: 1, where the MAD2019-H848A variant polypeptide comprises an alanine at position 848 according to SEQ ID NO: 1. In an aspect, this disclosure provides a MAD2019-H848A variant polypeptide comprising an amino acid sequence at least 85% identical or similar to SEQ ID NO: 1, where the MAD2019-H848A variant polypeptide comprises an alanine at position 848 according to SEQ ID NO: 1. In an aspect, this disclosure provides a MAD2019-H848A variant polypeptide comprising an amino acid sequence at least 90% identical or similar to SEQ ID NO: 1, where the MAD2019-H848A variant polypeptide comprises an alanine at position 848 according to SEQ ID NO: 1. In an aspect, this disclosure provides a MAD2019-H848A variant polypeptide comprising an amino acid sequence at least 92.5% identical or similar to SEQ ID NO: 1, where the MAD2019-H848A variant polypeptide comprises an alanine at position 848 according to SEQ ID NO: 1. In an aspect, this disclosure provides a MAD2019-H848A variant polypeptide comprising an amino acid sequence at least 95% identical or similar to SEQ ID NO: 1, where the MAD2019-H848A variant polypeptide comprises an alanine at position 848 according to SEQ ID NO: 1. In an aspect, this disclosure provides a MAD2019-H848A variant polypeptide comprising an amino acid sequence at least 96% identical or similar to SEQ ID NO: 1, where the MAD2019-H848A variant polypeptide comprises an alanine at position 848 according to SEQ ID NO: 1. In an aspect, this disclosure provides a MAD2019-H848A variant polypeptide comprising an amino acid sequence at least 97% identical or similar to SEQ ID NO: 1, where the MAD2019-H848A variant polypeptide comprises an alanine at position 848 according to SEQ ID NO: 1. In an aspect, this disclosure provides a MAD2019-H848A variant polypeptide comprising an amino acid sequence at least 98% identical or similar to SEQ ID NO: 1, where the MAD2019-H848A variant polypeptide comprises an alanine at position 848 according to SEQ ID NO: 1. In an aspect, this disclosure provides a MAD2019-H848A variant polypeptide comprising an amino acid sequence at least 99% identical or similar to SEQ ID NO: 1, where the MAD2019-H848A variant polypeptide comprises an alanine at position 848 according to SEQ ID NO: 1. In an aspect, this disclosure provides a MAD2019-H848A variant polypeptide comprising an amino acid sequence at least 99.5% identical or similar to SEQ ID NO: 1, where the MAD2019-H848A variant polypeptide comprises an alanine at position 848 according to SEQ ID NO: 1. In an aspect, this disclosure provides a MAD2019-H848A variant polypeptide comprising an amino acid sequence 100% similar to SEQ ID NO: 1, where the MAD2019-H848A variant polypeptide comprises an alanine at position 848 according to SEQ ID NO: 1. In an aspect, this disclosure provides a MAD2019-H848A variant polypeptide comprising the amino acid sequence of SEQ ID NO: 1.

The terms “percent identity” or “percent identical” as used herein in reference to two or more nucleotide or amino acid sequences is calculated by (i) comparing two optimally aligned sequences (nucleotide or amino acid) over a window of comparison (the “alignable” region or regions), (ii) determining the number of positions at which the identical nucleic acid base (for nucleotide sequences) or amino acid residue (for proteins and polypeptides) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

When percentage of sequence identity is used in reference to amino acids it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

For optimal alignment of sequences to calculate their percent identity, various pair-wise or multiple sequence alignment algorithms and programs are known in the art, such as ClustalW or Basic Local Alignment Search Tool® (BLAST™), etc., that can be used to compare the sequence identity or similarity between two or more nucleotide or amino acid sequences. Although other alignment and comparison methods are known in the art, the alignment and percent identity between two sequences (including the percent identity ranges described above) can be as determined by the ClustalW algorithm, see, e.g., Chenna et al., “Multiple sequence alignment with the Clustal series of programs,” Nucleic Acids Research 31:3497-3500 (2003); Thompson et al., “Clustal W: Improving the sensitivity of progressive multiple sequence alignment through sequence weighting, position-specific gap penalties and weight matrix choice,” Nucleic Acids Research 22:4673-4680 (1994); Larkin M A et al., “Clustal W and Clustal X version 2.0,” Bioinformatics 23:2947-48 (2007); and Altschul et al. “Basic local alignment search tool.” J. Mol. Biol. 215:403-410 (1990), the entire contents and disclosures of which are incorporated herein by reference.

The terms “percent complementarity” or “percent complementary” as used herein in reference to two nucleotide sequences is similar to the concept of percent identity but refers to the percentage of nucleotides of a query sequence that optimally base-pair or hybridize to nucleotides a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (e.g., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%.

The use of the term “polynucleotide” or “nucleic acid molecule” is not intended to limit the present disclosure to polynucleotides comprising deoxyribonucleic acid (DNA). For example, ribonucleic acid (RNA) molecules are also envisioned. Those of ordinary skill in the art will recognize that polynucleotides and nucleic acid molecules can comprise ribonucleotides and combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like. In an aspect, a nucleic acid molecule provided herein is a DNA molecule. In another aspect, a nucleic acid molecule provided herein is an RNA molecule. In an aspect, a nucleic acid molecule provided herein is single-stranded. In another aspect, a nucleic acid molecule provided herein is double-stranded. In an aspect, a nucleic acid molecule encodes a polypeptide.

In an aspect, this disclosure provides a nucleic acid molecule encoding any MAD2019-H848A variant polypeptide provided herein. In an aspect, this disclosure provides a nucleic acid molecule encoding any fusion protein provided herein. In an aspect, this disclosure provides a nucleic acid molecule encoding any reverse transcriptase provided herein. In an aspect, this disclosure provides a nucleic acid molecule encoding any guide provided herein. In an aspect, this disclosure provides a nucleic acid molecule encoding any homology arm provided herein.

In an aspect, any nucleic acid molecule, fusion protein, or MAD2019-H848A variant polypeptide provided herein is provided for use in vitro. In an aspect, any nucleic acid molecule, fusion protein, or MAD2019-H848A variant polypeptide provided herein is provided for use in vivo. In an aspect, any nucleic acid molecule, fusion protein, or MAD2019-H848A variant polypeptide provided herein is provided for use ex vivo.

In an aspect, a nucleic acid molecule comprises a promoter. In an aspect, a promoter is operably linked to a nucleic acid molecule encoding a MAD2019-H848A variant polypeptide. In an aspect, a promoter is operably linked to a nucleic acid molecule encoding a reverse transcriptase. In an aspect, a promoter is operably linked to a nucleic acid molecule encoding a fusion protein. In an aspect, a promoter is operably linked to a nucleic acid molecule encoding a guide. In an aspect, a promoter is operably linked to a nucleic acid molecule encoding a homology arm. Any promoter suitable for expression in a cell of interest can be used.

As commonly understood in the art, the term “promoter” refers to a DNA sequence that contains an RNA polymerase binding site, a transcription start site, and/or a TATA box and assists or promotes the transcription and expression of an associated transcribable polynucleotide sequence and/or gene (or transgene). A promoter can be synthetically produced, varied, or derived from a known or naturally occurring promoter sequence or other promoter sequence. A promoter can also include a chimeric promoter comprising a combination of two or more heterologous sequences. A promoter of the present application can thus include variants of promoter sequences that are similar in composition, but not identical to, other promoter sequence(s) known or provided herein.

As used herein, “operably linked” refers to a functional linkage between two or more elements. For example, an operable linkage between a polynucleotide of interest and a regulatory sequence (e.g., a promoter) is a functional link that allows for expression of the polynucleotide of interest. Operably linked elements may be contiguous or non-contiguous. In an aspect, a promoter is operably linked to a heterologous nucleic acid molecule.

In an aspect, a promoter is an inducible promoter. As used herein, an “inducible promoter” refers to a regulated promoter that becomes active (e.g., it drives the expression of an operably linked sequence) in a cell in response to a specific stimulus.

In an aspect, a promoter is a constitutive promoter. As used herein, a “constitutive promoter” refers to a promoter that is active in vivo at all times. Typically, the activity of a constitutive promoter is limited only by the presence of a suitable RNA polymerase at a suitable concentration.

In an aspect, a nucleic acid molecule comprises a transcription terminator. In an aspect, a transcription terminator is operably linked to a nucleic acid molecule encoding a MAD2019-H848A variant polypeptide. In an aspect, a transcription terminator is operably linked to a nucleic acid molecule encoding a reverse transcriptase. In an aspect, a transcription terminator is operably linked to a nucleic acid molecule encoding a fusion protein. In an aspect, a transcription terminator is operably linked to a nucleic acid molecule encoding a guide. In an aspect, a transcription terminator is operably linked to a nucleic acid molecule encoding a homology arm. Any transcription terminator suitable for terminating transcription of a nucleic acid molecule in a cell of interest can be used.

As used herein, the term “polypeptide” refers to a chain of at least two covalently linked amino acids. Polypeptides can be encoded by polynucleotides provided herein. Proteins provided herein can be encoded by nucleic acid molecules provided herein. Proteins can comprise polypeptides provided herein. As used herein, a “protein” refers to a chain of amino acid residues that is capable of providing structure or enzymatic activity to a cell. In an aspect, a MAD2019-H848A variant polypeptide is a protein.

In an aspect, a MAD2019-H848A variant polypeptide comprises a threonine to glycine amino acid substitution at position 67 (T67G) as compared to SEQ ID NO: 1.

In an aspect, a MAD2019-H848A variant polypeptide comprises a serine to arginine amino acid substitution at position 409 (S409R) as compared to SEQ ID NO: 1.

In an aspect, a MAD2019-H848A variant polypeptide comprises a leucine to lysine amino acid substitution at position 500 (L500K) as compared to SEQ ID NO: 1.

In an aspect, a MAD2019-H848A variant polypeptide comprises a leucine to arginine amino acid substitution at position 500 (L500R) as compared to SEQ ID NO: 1.

In an aspect, a MAD2019-H848A variant polypeptide comprises a glycine to phenylalanine amino acid substitution at position 578 (G578F) as compared to SEQ ID NO: 1.

In an aspect, a MAD2019-H848A variant polypeptide comprises a leucine to glutamine amino acid substitution at position 624 (L624Q) as compared to SEQ ID NO: 1.

In an aspect, a MAD2019-H848A variant polypeptide comprises an asparagine to serine amino acid substitution at position 669 (N669S) as compared to SEQ ID NO: 1.

In an aspect, a MAD2019-H848A variant polypeptide comprises an aspartic acid to alanine amino acid substitution at position 700 (D700A) as compared to SEQ ID NO: 1.

In an aspect, a MAD2019-H848A variant polypeptide comprises an aspartic acid to proline amino acid substitution at position 701 (D701P) as compared to SEQ ID NO: 1.

In an aspect, a MAD2019-H848A variant polypeptide comprises an aspartic acid to asparagine amino acid substitution at position 701 (D701N) as compared to SEQ ID NO: 1.

In an aspect, a MAD2019-H848A variant polypeptide comprises an aspartic acid to threonine amino acid substitution at position 701 (D701T) as compared to SEQ ID NO: 1.

In an aspect, a MAD2019-H848A variant polypeptide comprises a lysine to serine amino acid substitution at position 720 (K720S) as compared to SEQ ID NO: 1.

In an aspect, a MAD2019-H848A variant polypeptide comprises a leucine to arginine amino acid substitution at position 1110 (L1110R) as compared to SEQ ID NO: 1.

In an aspect, a MAD2019-H848A variant polypeptide comprises an isoleucine to arginine amino acid substitution at position 1142 (I1142R) as compared to SEQ ID NO: 1.

In an aspect, a MAD2019-H848A variant polypeptide comprises an isoleucine to lysine amino acid substitution at position 1142 (I1142K) as compared to SEQ ID NO: 1.

In an aspect, a MAD2019-H848A variant polypeptide comprises a valine to threonine amino acid substitution at position 1143 (V1143T) as compared to SEQ ID NO: 1.

In an aspect, a MAD2019-H848A variant polypeptide comprises an alanine to histidine amino acid substitution at position 1221 (A1221H) as compared to SEQ ID NO: 1.

In an aspect, a MAD2019-H848A variant polypeptide comprises a lysine to arginine amino acid substitution at position 1285 (K1285R) as compared to SEQ ID NO: 1.

In an aspect, a MAD2019-H848A variant polypeptide comprises an alanine to arginine amino acid substitution at position 1321 (A1321R) as compared to SEQ ID NO: 1.

In an aspect, a MAD2019-H848A variant polypeptide comprises an alanine to lysine amino acid substitution at position 1321 (A1321K) as compared to SEQ ID NO: 1.

In an aspect, a MAD2019-H848A variant polypeptide comprises a serine to glutamine amino acid substitution at position 1336 (S1336Q) as compared to SEQ ID NO: 1.

In an aspect, a MAD2019-H848A variant polypeptide comprises an alanine to arginine amino acid substitution at position 1339 (A1339R) as compared to SEQ ID NO: 1.

In an aspect, a MAD2019-H848A variant polypeptide comprises an amino acid substitution selected from the group consisting of T67G, S409R, L500K, L500R, G578F, L624Q, N669S, D700A, D701P, D701N, D701T, K720S, L1110R, D1139N, I1142R, I1142K, V1143T, A1221H, K1285R, A1321R, A1321K, S1136Q, and A1139R as compared to SEQ ID NO: 1. In an aspect, a MAD2019-H848A variant polypeptide comprises at least two amino acid substitutions selected from the group consisting of T67G, S409R, L500K, L500R, G578F, L624Q, N669S, D700A, D701P, D701N, D701T, K720S, L1110R, D1139N, I1142R, I1142K, V1143T, A1221H, K1285R, A1321R, A1321K, S1136Q, and A1139R as compared to SEQ ID NO: 1. In an aspect, a MAD2019-H848A variant polypeptide comprises at least three amino acid substitutions selected from the group consisting of T67G, S409R, L500K, L500R, G578F, L624Q, N669S, D700A, D701P, D701N, D701T, K720S, L1110R, D1139N, I1142R, I1142K, V1143T, A1221H, K1285R, A1321R, A1321K, S1136Q, and A1139R as compared to SEQ ID NO: 1. In an aspect, a MAD2019-H848A variant polypeptide comprises at least four amino acid substitutions selected from the group consisting of T67G, S409R, L500K, L500R, G578F, L624Q, N669S, D700A, D701P, D701N, D701T, K720S, L1110R, D1139N, I1142R, I1142K, V1143T, A1221H, K1285R, A1321R, A1321K, S1136Q, and A1139R as compared to SEQ ID NO: 1. In an aspect, a MAD2019-H848A variant polypeptide comprises at least five amino acid substitutions selected from the group consisting of T67G, S409R, L500K, L500R, G578F, L624Q, N669S, D700A, D701P, D701N, D701T, K720S, L1110R, D1139N, I1142R, I1142K, V1143T, A1221H, K1285R, A1321R, A1321K, S1136Q, and A1139R as compared to SEQ ID NO: 1. In an aspect, a MAD2019-H848A variant polypeptide comprises at least six amino acid substitutions selected from the group consisting of T67G, S409R, L500K, L500R, G578F, L624Q, N669S, D700A, D701P, D701N, D701T, K720S, L1110R, D1139N, I1142R, I1142K, V1143T, A1221H, K1285R, A1321R, A1321K, S1136Q, and A1139R as compared to SEQ ID NO: 1. In an aspect, a MAD2019-H848A variant polypeptide comprises at least seven amino acid substitutions selected from the group consisting of T67G, S409R, L500K, L500R, G578F, L624Q, N669S, D700A, D701P, D701N, D701T, K720S, L1110R, D1139N, I1142R, I1142K, V1143T, A1221H, K1285R, A1321R, A1321K, S1136Q, and A1139R as compared to SEQ ID NO: 1. In an aspect, a MAD2019-H848A variant polypeptide comprises at least eight amino acid substitutions selected from the group consisting of T67G, S409R, L500K, L500R, G578F, L624Q, N669S, D700A, D701P, D701N, D701T, K720S, L1110R, D1139N, I1142R, I1142K, V1143T, A1221H, K1285R, A1321R, A1321K, S1136Q, and A1139R as compared to SEQ ID NO: 1.

In an aspect, a MAD2019-H848A variant polypeptide comprises an L500R amino acid substitution, a D700A amino acid substitution, a D701P amino acid substitution, a K720S amino acid substitution, an I1142K amino acid substitution, and a V1143T amino acid substitution as compared to SEQ ID NO: 1.

In an aspect, a MAD2019-H848A variant polypeptide comprises an L500R amino acid substitution, a D700A amino acid substitution, a D701P amino acid substitution, a K720S amino acid substitution, and a V1143T amino acid substitution as compared to SEQ ID NO: 1.

In an aspect, a MAD2019-H848A variant polypeptide comprises an L500K amino acid substitution and a V1143T amino acid substitution as compared to SEQ ID NO: 1.

In an aspect, a MAD2019-H848A variant polypeptide comprises an S409R amino acid substitution, an L500K amino acid substitution, and a V1143T amino acid substitution as compared to SEQ ID NO: 1.

In an aspect, a MAD2019-H848A variant polypeptide comprises a V1143T amino acid substitution and an A1221H amino acid substitution as compared to SEQ ID NO: 1.

In an aspect, a MAD2019-H848A variant polypeptide comprises an L500K amino acid substitution, a V1143T amino acid substitution, and an A1221H amino acid substitution as compared to SEQ ID NO: 1.

In an aspect, a MAD2019-H848A variant polypeptide comprises an L500K amino acid substitution, an I1142R amino acid substitution, a V1143T amino acid substitution, and an A1221H amino acid substitution as compared to SEQ ID NO: 1.

In an aspect, a MAD2019-H848A variant polypeptide comprises an L500K amino acid substitution, a D1139N amino acid substitution, a V1143T amino acid substitution, and an A1221H amino acid substitution as compared to SEQ ID NO: 1.

In an aspect, a MAD2019-H848A variant polypeptide comprises an L500K amino acid substitution, a V1143T amino acid substitution, an A1221H amino acid substitution, and a K1285R amino acid substitution as compared to SEQ ID NO: 1.

In an aspect, a MAD2019-H848A variant polypeptide comprises improved nicking efficiency as compared to SEQ ID NO: 1.

In an aspect, a MAD2019-H848A variant polypeptide nicks a double-stranded DNA molecule at an efficiency that is within 1% of the nicking efficiency of SEQ ID NO: 1. In an aspect, a MAD2019-H848A variant polypeptide nicks a double-stranded DNA molecule at an efficiency that is within 2.5% of the nicking efficiency of SEQ ID NO: 1. In an aspect, a MAD2019-H848A variant polypeptide nicks a double-stranded DNA molecule at an efficiency that is within 5% of the nicking efficiency of SEQ ID NO: 1. In an aspect, a MAD2019-H848A variant polypeptide nicks a double-stranded DNA molecule at an efficiency that is within 7.5% of the nicking efficiency of SEQ ID NO: 1. In an aspect, a MAD2019-H848A variant polypeptide nicks a double-stranded DNA molecule at an efficiency that is within 10% of the nicking efficiency of SEQ ID NO: 1. In an aspect, a MAD2019-H848A variant polypeptide nicks a double-stranded DNA molecule at an efficiency that is within 12.5% of the nicking efficiency of SEQ ID NO: 1. In an aspect, a MAD2019-H848A variant polypeptide nicks a double-stranded DNA molecule at an efficiency that is within 15% of the nicking efficiency of SEQ ID NO: 1. In an aspect, a MAD2019-H848A variant polypeptide nicks a double-stranded DNA molecule at an efficiency that is within 20% of the nicking efficiency of SEQ ID NO: 1. In an aspect, a MAD2019-H848A variant polypeptide nicks a double-stranded DNA molecule at an efficiency that is within 25% of the nicking efficiency of SEQ ID NO: 1. In an aspect, a MAD2019-H848A variant polypeptide nicks a double-stranded DNA molecule at an efficiency that is within 30% of the nicking efficiency of SEQ ID NO: 1. In an aspect, a MAD2019-H848A variant polypeptide nicks a double-stranded DNA molecule at an efficiency that is within 35% of the nicking efficiency of SEQ ID NO: 1. In an aspect, a MAD2019-H848A variant polypeptide nicks a double-stranded DNA molecule at an efficiency that is within 40% of the nicking efficiency of SEQ ID NO: 1. In an aspect, a MAD2019-H848A variant polypeptide nicks a double-stranded DNA molecule at an efficiency that is within 45% of the nicking efficiency of SEQ ID NO: 1. In an aspect, a MAD2019-H848A variant polypeptide nicks a double-stranded DNA molecule at an efficiency that is within 50% of the nicking efficiency of SEQ ID NO: 1. In an aspect, a MAD2019-H848A variant polypeptide nicks a double-stranded DNA molecule at an efficiency that is within 55% of the nicking efficiency of SEQ ID NO: 1. In an aspect, a MAD2019-H848A variant polypeptide nicks a double-stranded DNA molecule at an efficiency that is within 60% of the nicking efficiency of SEQ ID NO: 1. In an aspect, a MAD2019-H848A variant polypeptide nicks a double-stranded DNA molecule at an efficiency that is within 65% of the nicking efficiency of SEQ ID NO: 1. In an aspect, a MAD2019-H848A variant polypeptide nicks a double-stranded DNA molecule at an efficiency that is within 70% of the nicking efficiency of SEQ ID NO: 1. In an aspect, a MAD2019-H848A variant polypeptide nicks a double-stranded DNA molecule at an efficiency that is within 75% of the nicking efficiency of SEQ ID NO: 1. In an aspect, a MAD2019-H848A variant polypeptide nicks a double-stranded DNA molecule at an efficiency that is within 80% of the nicking efficiency of SEQ ID NO: 1. In an aspect, a MAD2019-H848A variant polypeptide nicks a double-stranded DNA molecule at an efficiency that is within 90% of the nicking efficiency of SEQ ID NO: 1.

In an aspect, a MAD2019-H848A variant polypeptide nicks a double-stranded DNA molecule at an efficiency that is between 1% and 90% of the nicking efficiency of SEQ ID NO: 1. In an aspect, a MAD2019-H848A variant polypeptide nicks a double-stranded DNA molecule at an efficiency that is between 1% and 75% of the nicking efficiency of SEQ ID NO: 1. In an aspect, a MAD2019-H848A variant polypeptide nicks a double-stranded DNA molecule at an efficiency that is between 1% and 60% of the nicking efficiency of SEQ ID NO: 1. In an aspect, a MAD2019-H848A variant polypeptide nicks a double-stranded DNA molecule at an efficiency that is between 1% and 50% of the nicking efficiency of SEQ ID NO: 1. In an aspect, a MAD2019-H848A variant polypeptide nicks a double-stranded DNA molecule at an efficiency that is between 1% and 40% of the nicking efficiency of SEQ ID NO: 1. In an aspect, a MAD2019-H848A variant polypeptide nicks a double-stranded DNA molecule at an efficiency that is between 1% and 30% of the nicking efficiency of SEQ ID NO: 1. In an aspect, a MAD2019-H848A variant polypeptide nicks a double-stranded DNA molecule at an efficiency that is between 1% and 20% of the nicking efficiency of SEQ ID NO: 1. In an aspect, a MAD2019-H848A variant polypeptide nicks a double-stranded DNA molecule at an efficiency that is between 1% and 10% of the nicking efficiency of SEQ ID NO: 1. In an aspect, a MAD2019-H848A variant polypeptide nicks a double-stranded DNA molecule at an efficiency that is between 10% and 70% of the nicking efficiency of SEQ ID NO: 1. In an aspect, a MAD2019-H848A variant polypeptide nicks a double-stranded DNA molecule at an efficiency that is between 10% and 60% of the nicking efficiency of SEQ ID NO: 1. In an aspect, a MAD2019-H848A variant polypeptide nicks a double-stranded DNA molecule at an efficiency that is between 10% and 50% of the nicking efficiency of SEQ ID NO: 1. In an aspect, a MAD2019-H848A variant polypeptide nicks a double-stranded DNA molecule at an efficiency that is between 1% and 25% of the nicking efficiency of SEQ ID NO: 1.

Nicking efficiency can be measured using any suitable method in the art. For example, see Wei et al., Scientific Reports, 6:32560 (2016).

In an aspect, a MAD2019-H848A variant polypeptide further comprises at least one nuclear localization signal (NLS). In an aspect, a fusion protein comprises at least one NLS. Nuclear localization signals are known in the art as short (e.g., without being limiting, typically fewer than 25 amino acids) amino acid sequences that “tag” proteins for import into a cell's nucleus via nuclear transport. In an aspect, a MAD2019-H848A variant polypeptide comprises at least two NLSs. In an aspect, a MAD2019-H848A variant polypeptide comprises at least three NLSs. In an aspect, a MAD2019-H848A variant polypeptide comprises at least four NLSs. In an aspect, a MAD2019-H848A variant polypeptide comprises at least five NLSs. In an aspect, a MAD2019-H848A variant polypeptide comprises at least six NLSs. In an aspect, a MAD2019-H848A variant polypeptide comprises at least seven NLSs. In an aspect, a MAD2019-H848A variant polypeptide comprises at least eight NLSs. In an aspect, a MAD2019-H848A variant polypeptide comprises at least nine NLSs. In an aspect, a MAD2019-H848A variant polypeptide comprises at least ten NLSs.

In an aspect, an NLS is positioned before the N-terminus of a MAD2019-H848A variant polypeptide. In an aspect, an NLS is positioned after the C-terminus of a MAD2019-H848A variant polypeptide. In an aspect, a MAD2019-H848A variant polypeptide comprises a first NLS before its N-terminus and a second NLS after its C-terminus.

In an aspect, an NLS is positioned before the N-terminus of a fusion protein. In an aspect, an NLS is positioned after the C-terminus of a fusion protein. In an aspect, a fusion protein comprises a first NLS before its N-terminus and a second NLS after its C-terminus.

In an aspect, an NLS comprises equal to or fewer than 50 amino acids. In an aspect, an NLS comprises equal to or fewer than 40 amino acids. In an aspect, an NLS comprises equal to or fewer than 30 amino acids. In an aspect, an NLS comprises equal to or fewer than 25 amino acids. In an aspect, an NLS comprises equal to or fewer than 20 amino acids. In an aspect, an NLS comprises equal to or fewer than 15 amino acids. In an aspect, an NLS comprises equal to or fewer than 10 amino acids.

Any suitable NLS can be used with the MAD2019-H848A variant polypeptides provided herein. In an aspect, an NLS comprises an amino acid sequence at least 70% identical or similar to SEQ ID Nos: 15 or 19. In an aspect, an NLS comprises an amino acid sequence at least 80% identical or similar to SEQ ID Nos: 15 or 19. In an aspect, an NLS comprises an amino acid sequence at least 85% identical or similar to SEQ ID Nos: 15 or 19. In an aspect, an NLS comprises an amino acid sequence at least 90% identical or similar to SEQ ID Nos: 15 or 19. In an aspect, an NLS comprises an amino acid sequence at least 92.5% identical or similar to SEQ ID Nos: 15 or 19. In an aspect, an NLS comprises an amino acid sequence at least 95% identical or similar to SEQ ID Nos: 15 or 19. In an aspect, an NLS comprises an amino acid sequence at least 97.5% identical or similar to SEQ ID Nos: 15 or 19. In an aspect, an NLS comprises an amino acid sequence at least 99% identical or similar to SEQ ID Nos: 15 or 19. In an aspect, an NLS comprises an amino acid sequence selected from the group consisting of SEQ ID Nos: 15 and 19.

In an aspect, this disclosure provides a nucleic acid sequence that encodes an NLS.

In an aspect, this disclosure provides a nucleic acid sequence encoding any MAD2019-H848A variant polypeptide provided herein. In an aspect, this disclosure provides a nucleic acid sequence encoding the amino acid sequence of any one of SEQ ID Nos: 1, 12, 13, and 15 to 23. In an aspect, this disclosure provides a nucleic acid sequence encoding an amino acid sequence at least 70% identical or similar to an amino acid sequence selected from the group consisting of SEQ ID Nos: 1, 12, 13, and 15 to 23. In an aspect, this disclosure provides a nucleic acid sequence encoding an amino acid sequence at least 75% identical or similar to an amino acid sequence selected from the group consisting of SEQ ID Nos: 1, 12, 13, and 15 to 23. In an aspect, this disclosure provides a nucleic acid sequence encoding an amino acid sequence at least 80% identical or similar to an amino acid sequence selected from the group consisting of SEQ ID Nos: 1, 12, 13, and 15 to 23. In an aspect, this disclosure provides a nucleic acid sequence encoding an amino acid sequence at least 85% identical or similar to an amino acid sequence selected from the group consisting of SEQ ID Nos: 1, 12, 13, and 15 to 23. In an aspect, this disclosure provides a nucleic acid sequence encoding an amino acid sequence at least 90% identical or similar to an amino acid sequence selected from the group consisting of SEQ ID Nos: 1, 12, 13, and 15 to 23. In an aspect, this disclosure provides a nucleic acid sequence encoding an amino acid sequence at least 92.5% identical or similar to an amino acid sequence selected from the group consisting of SEQ ID Nos: 1, 12, 13, and 15 to 23. In an aspect, this disclosure provides a nucleic acid sequence encoding an amino acid sequence at least 95% identical or similar to an amino acid sequence selected from the group consisting of SEQ ID Nos: 1, 12, 13, and 15 to 23. In an aspect, this disclosure provides a nucleic acid sequence encoding an amino acid sequence at least 97.5% identical or similar to an amino acid sequence selected from the group consisting of SEQ ID Nos: 1, 12, 13, and 15 to 23. In an aspect, this disclosure provides a nucleic acid sequence encoding an amino acid sequence at least 99% identical or similar to an amino acid sequence selected from the group consisting of SEQ ID Nos: 1, 12, 13, and 15 to 23. In an aspect, this disclosure provides a nucleic acid sequence encoding an amino acid sequence at least 99.5% identical or similar to an amino acid sequence selected from the group consisting of SEQ ID Nos: 1, 12, 13, and 15 to 23. In an aspect, this disclosure provides a nucleic acid sequence encoding an amino acid sequence at least 100% similar to an amino acid sequence selected from the group consisting of SEQ ID Nos: 1, 12, 13, and 15 to 23.

In an aspect, this disclosure provides a nucleoprotein complex comprising any of the MAD2019-H848A variant polypeptides provided herein and a nucleic acid molecule. In an aspect, this disclosure provides a nucleoprotein complex comprising any of the fusion proteins provided herein and a nucleic acid molecule. As used herein, a “nucleoprotein complex” refers to a protein conjugated with a nucleic acid molecule. When a nucleoprotein complex comprises an RNA molecule, it can be referred to as a ribonucleoprotein complex. When a nucleoprotein complex comprises a DNA molecule, it can be referred to as a deoxyribonucleoprotein complex. In an aspect, a nucleoprotein complex provided herein is a ribonucleoprotein complex. In an aspect, a nucleoprotein complex provided herein is a deoxyribonucleoprotein complex. In an aspect, the nucleic acid molecule component of a nucleoprotein complex is an RNA molecule. In an aspect, the nucleic acid molecule component of a nucleoprotein complex is an DNA molecule.

In an aspect, a nucleic acid molecule provided herein encodes a guide. In an aspect, a nucleic acid molecule provided herein comprises a guide. As used herein, a “guide” refers to a nucleic acid molecule that is capable of guiding a protein it is complexed with to a target nucleic acid molecule. Typically, a guide is complementary to a target nucleic acid molecule, although perfect (e.g., 100%) complementarity is not required, and a guide can hybridize with the target nucleic acid molecule. In an aspect, a guide is a DNA molecule. In an aspect, a guide is an RNA molecule. In an aspect, a guide comprises a DNA molecule and an RNA molecule. In an aspect, a guide is single-stranded. In an aspect, a guide is double-stranded. In an aspect, a guide comprises one or more sections that are single-stranded and one or more regions that are double-stranded.

When a guide is an RNA molecule, it can be referred to as a “guide RNA” or “gRNA.” In an aspect, a nucleoprotein complex or a ribonucleoprotein complex comprises a gRNA. In an aspect, a gRNA is capable of guiding a MAD2019-H848A variant polypeptide to a target nucleic acid molecule. In an aspect, a gRNA guides a MAD2019-H848A variant polypeptide to a target nucleic acid molecule. In an aspect, a gRNA is capable of guiding a fusion protein to a target nucleic acid molecule. In an aspect, a gRNA guides a fusion protein to a target nucleic acid molecule.

In an aspect, a nucleoprotein complex comprises a MAD2019-H848A variant polypeptide and a gRNA. In an aspect, a nucleoprotein complex comprises a fusion protein and a gRNA. In an aspect, a nucleoprotein complex comprises a MAD2019-H848A variant polypeptide and a guide. In an aspect, a nucleoprotein complex comprises a fusion protein and a guide. In an aspect, a nucleoprotein complex comprises a MAD2019-H848A variant polypeptide, a homology arm, and a gRNA. In an aspect, a nucleoprotein complex comprises a fusion protein, a homology arm, and a gRNA. In an aspect, a nucleoprotein complex comprises a MAD2019-H848A variant polypeptide, a homology arm, and a guide. In an aspect, a nucleoprotein complex comprises a fusion protein, a homology arm, and a guide.

In an aspect, a guide comprises at least 5 nucleotides. In an aspect, a guide comprises at least 10 nucleotides. In an aspect, a guide comprises at least 15 nucleotides. In an aspect, a guide comprises at least 20 nucleotides. In an aspect, a guide comprises at least 25 nucleotides. In an aspect, a guide comprises at least 30 nucleotides. In an aspect, a guide comprises at least 35 nucleotides. In an aspect, a guide comprises at least 40 nucleotides. In an aspect, a guide comprises at least 45 nucleotides. In an aspect, a guide comprises at least 50 nucleotides. In an aspect, a guide comprises at least 60 nucleotides. In an aspect, a guide comprises at least 70 nucleotides. In an aspect, a guide comprises at least 80 nucleotides. In an aspect, a guide comprises at least 90 nucleotides. In an aspect, a guide comprises at least 100 nucleotides. In an aspect, a guide comprises at least 125 nucleotides.

In an aspect, a guide comprises between 5 nucleotides and 150 nucleotides. In an aspect, a guide comprises between 5 nucleotides and 125 nucleotides. In an aspect, a guide comprises between 5 nucleotides and 100 nucleotides. In an aspect, a guide comprises between 5 nucleotides and 75 nucleotides. In an aspect, a guide comprises between 5 nucleotides and 50 nucleotides. In an aspect, a guide comprises between 5 nucleotides and 40 nucleotides. In an aspect, a guide comprises between 5 nucleotides and 30 nucleotides. In an aspect, a guide comprises between 5 nucleotides and 25 nucleotides. In an aspect, a guide comprises between 15 nucleotides and 30 nucleotides. In an aspect, a guide comprises between 15 nucleotides and 25 nucleotides. In an aspect, a guide comprises between 20 nucleotides and 150 nucleotides. In an aspect, a guide comprises between 20 nucleotides and 125 nucleotides. In an aspect, a guide comprises between 20 nucleotides and 100 nucleotides. In an aspect, a guide comprises between 20 nucleotides and 75 nucleotides. In an aspect, a guide comprises between 20 nucleotides and 50 nucleotides. In an aspect, a guide comprises between 40 nucleotides and 100 nucleotides. In an aspect, a guide comprises between 50 nucleotides and 150 nucleotides. In an aspect, a guide comprises between 50 nucleotides and 100 nucleotides.

In an aspect, there are zero mismatches between a guide or gRNA and a target nucleic acid molecule when optimally aligned. In an aspect, there is no more than 1 mismatch between a guide or gRNA and a target nucleic acid molecule when optimally aligned. In an aspect, there are no more than 2 mismatches between a guide or gRNA and a target nucleic acid molecule when optimally aligned. In an aspect, there are no more than 3 mismatches between a guide or gRNA and a target nucleic acid molecule when optimally aligned. In an aspect, there are no more than 4 mismatches between a guide or gRNA and a target nucleic acid molecule when optimally aligned. In an aspect, there are no more than 5 mismatches between a guide or gRNA and a target nucleic acid molecule when optimally aligned. In an aspect, there are no more than 6 mismatches between a guide or gRNA and a target nucleic acid molecule when optimally aligned. In an aspect, there are no more than 7 mismatches between a guide or gRNA and a target nucleic acid molecule when optimally aligned. In an aspect, there are no more than 8 mismatches between a guide or gRNA and a target nucleic acid molecule when optimally aligned. In an aspect, there are no more than 9 mismatches between a guide or gRNA and a target nucleic acid molecule when optimally aligned. In an aspect, there are no more than 10 mismatches between a guide or gRNA and a target nucleic acid molecule when optimally aligned. In an aspect, there are no more than 15 mismatches between a guide or gRNA and a target nucleic acid molecule when optimally aligned.

In an aspect, a guide forms a nucleoprotein complex with a MAD2019-H848A variant polypeptide within a cell. In an aspect, a gRNA forms a nucleoprotein complex with a MAD2019-H848A variant polypeptide within a cell. In an aspect, a guide forms a nucleoprotein complex with a fusion protein within a cell. In an aspect, a gRNA forms a nucleoprotein complex with a fusion protein within a cell.

In an aspect, a guide forms a nucleoprotein complex with a MAD2019-H848A variant polypeptide. In an aspect, a gRNA forms a nucleoprotein complex with a MAD2019-H848A variant polypeptide. In an aspect, a guide forms a nucleoprotein complex with a fusion protein. In an aspect, a gRNA forms a nucleoprotein complex with a fusion protein.

In an aspect, a nucleoprotein complex comprises a fusion protein and a gRNA, where the gRNA comprises a scaffold region comprising a nucleic acid sequence at least 80% identical to SEQ ID NO: 24. In an aspect, a nucleoprotein complex comprises a fusion protein and a gRNA, where the gRNA comprises a scaffold region comprising a nucleic acid sequence at least 85% identical to SEQ ID NO: 24. In an aspect, a nucleoprotein complex comprises a fusion protein and a gRNA, where the gRNA comprises a scaffold region comprising a nucleic acid sequence at least 90% identical to SEQ ID NO: 24. In an aspect, a nucleoprotein complex comprises a fusion protein and a gRNA, where the gRNA comprises a scaffold region comprising a nucleic acid sequence at least 92.5% identical to SEQ ID NO: 24. In an aspect, a nucleoprotein complex comprises a fusion protein and a gRNA, where the gRNA comprises a scaffold region comprising a nucleic acid sequence at least 95% identical to SEQ ID NO: 24. In an aspect, a nucleoprotein complex comprises a fusion protein and a gRNA, where the gRNA comprises a scaffold region comprising a nucleic acid sequence at least 97.5% identical to SEQ ID NO: 24. In an aspect, a nucleoprotein complex comprises a fusion protein and a gRNA, where the gRNA comprises a scaffold region comprising a nucleic acid sequence 100% identical to SEQ ID NO: 24.

In an aspect, a nucleoprotein complex comprises a MAD2019-H848A variant polypeptide and a gRNA, where the gRNA comprises a scaffold region comprising a nucleic acid sequence at least 80% identical to SEQ ID NO: 24. In an aspect, a nucleoprotein complex comprises a MAD2019-H848A variant polypeptide and a gRNA, where the gRNA comprises a scaffold region comprising a nucleic acid sequence at least 85% identical to SEQ ID NO: 24. In an aspect, a nucleoprotein complex comprises a MAD2019-H848A variant polypeptide and a gRNA, where the gRNA comprises a scaffold region comprising a nucleic acid sequence at least 90% identical to SEQ ID NO: 24. In an aspect, a nucleoprotein complex comprises a MAD2019-H848A variant polypeptide and a gRNA, where the gRNA comprises a scaffold region comprising a nucleic acid sequence at least 92.5% identical to SEQ ID NO: 24. In an aspect, a nucleoprotein complex comprises a MAD2019-H848A variant polypeptide and a gRNA, where the gRNA comprises a scaffold region comprising a nucleic acid sequence at least 95% identical to SEQ ID NO: 24. In an aspect, a nucleoprotein complex comprises a MAD2019-H848A variant polypeptide and a gRNA, where the gRNA comprises a scaffold region comprising a nucleic acid sequence at least 97.5% identical to SEQ ID NO: 24. In an aspect, a nucleoprotein complex comprises a MAD2019-H848A variant polypeptide and a gRNA, where the gRNA comprises a scaffold region comprising a nucleic acid sequence 100% identical to SEQ ID NO: 24.

In an aspect, a nucleoprotein complex comprises a nickase and a gRNA, where the gRNA comprises a scaffold region comprising a nucleic acid sequence at least 80% identical to SEQ ID NO: 24. In an aspect, a nucleoprotein complex comprises a nickase and a gRNA, where the gRNA comprises a scaffold region comprising a nucleic acid sequence at least 85% identical to SEQ ID NO: 24. In an aspect, a nucleoprotein complex comprises a nickase and a gRNA, where the gRNA comprises a scaffold region comprising a nucleic acid sequence at least 90% identical to SEQ ID NO: 24. In an aspect, a nucleoprotein complex comprises a nickase and a gRNA, where the gRNA comprises a scaffold region comprising a nucleic acid sequence at least 92.5% identical to SEQ ID NO: 24. In an aspect, a nucleoprotein complex comprises a nickase and a gRNA, where the gRNA comprises a scaffold region comprising a nucleic acid sequence at least 95% identical to SEQ ID NO: 24. In an aspect, a nucleoprotein complex comprises a nickase and a gRNA, where the gRNA comprises a scaffold region comprising a nucleic acid sequence at least 97.5% identical to SEQ ID NO: 24. In an aspect, a nucleoprotein complex comprises a nickase and a gRNA, where the gRNA comprises a scaffold region comprising a nucleic acid sequence 100% identical to SEQ ID NO: 24.

In an aspect, a nucleoprotein complex comprises a gRNA, where the gRNA comprises a scaffold region comprising a nucleic acid sequence at least 80% identical to SEQ ID NO: 24. In an aspect, a nucleoprotein complex comprises a gRNA, where the gRNA comprises a scaffold region comprising a nucleic acid sequence at least 85% identical to SEQ ID NO: 24. In an aspect, a nucleoprotein complex comprises a gRNA, where the gRNA comprises a scaffold region comprising a nucleic acid sequence at least 90% identical to SEQ ID NO: 24. In an aspect, a nucleoprotein complex comprises a gRNA, where the gRNA comprises a scaffold region comprising a nucleic acid sequence at least 92.5% identical to SEQ ID NO: 24. In an aspect, a nucleoprotein complex comprises a gRNA, where the gRNA comprises a scaffold region comprising a nucleic acid sequence at least 95% identical to SEQ ID NO: 24. In an aspect, a nucleoprotein complex comprises a gRNA, where the gRNA comprises a scaffold region comprising a nucleic acid sequence at least 97.5% identical to SEQ ID NO: 24. In an aspect, a nucleoprotein complex comprises a gRNA, where the gRNA comprises a scaffold region comprising a nucleic acid sequence 100% identical to SEQ ID NO: 24.

In an aspect, a gRNA provided herein comprises at least one stem-and-loop structure. In an aspect, a gRNA provided herein is capable of binding to both strands of a target nucleic acid molecule. In an aspect, a gRNA provided herein comprises a reverse transcriptase template. In an aspect, a gRNA provided herein comprises an edit that is desired to be integrated into the target nucleic acid molecule. In an aspect, a gRNA provided herein comprises a primer binding site region. In an aspect, a gRNA provided herein comprises a spacer region. In an aspect, a gRNA provided herein comprises a scaffold region.

As used herein, a “spacer region” refers to a subsection of a gRNA that hybridizes to the strand of a target nucleic acid molecule that is not cut by a MAD2019-H848A variant polypeptide provided herein. As used herein, a “scaffold region” refers to a gRNA region that is positioned between a spacer region and a reverse transcriptase template. In an aspect, a scaffold region comprises at least one stem-and-loop structure. In an aspect, a scaffold region comprises at least two stem-and-loop structures. Without being limited by any scientific theory, a scaffold region is capable of interacting or complexing with a protein (e.g., a MAD2019-H848A variant polypeptide). As used herein, a “primer binding site region” refers to a subsection of gRNA that hybridizes to the strand of a target nucleic acid molecule that is cut by a MAD2019-H848A variant polypeptide provided herein. Typically, a MAD2019-H848A variant polypeptide will nick a target nucleic acid molecule downstream of the primer binding site region. The primer binding site region is immediately upstream of the reverse transcriptase template, which itself is upstream of the scaffold region. In an aspect, a reverse transcriptase reverse transcribes the reverse transcriptase template. In an aspect, a reverse transcriptase template comprises at least one edit that is desired to be integrated into a target nucleic acid molecule.

In an aspect, a gRNA comprises a scaffold region having a nucleic acid sequence at least 80% identical to SEQ ID NO: 24. In an aspect, a gRNA comprises a scaffold region having a nucleic acid sequence at least 82.5% identical to SEQ ID NO: 24. In an aspect, a gRNA comprises a scaffold region having a nucleic acid sequence at least 85% identical to SEQ ID NO: 24. In an aspect, a gRNA comprises a scaffold region having a nucleic acid sequence at least 87.5% identical to SEQ ID NO: 24. In an aspect, a gRNA comprises a scaffold region having a nucleic acid sequence at least 90% identical to SEQ ID NO: 24. In an aspect, a gRNA comprises a scaffold region having a nucleic acid sequence at least 91% identical to SEQ ID NO: 24. In an aspect, a gRNA comprises a scaffold region having a nucleic acid sequence at least 92% identical to SEQ ID NO: 24. In an aspect, a gRNA comprises a scaffold region having a nucleic acid sequence at least 93% identical to SEQ ID NO: 24. In an aspect, a gRNA comprises a scaffold region having a nucleic acid sequence at least 94% identical to SEQ ID NO: 24. In an aspect, a gRNA comprises a scaffold region having a nucleic acid sequence at least 95% identical to SEQ ID NO: 24. In an aspect, a gRNA comprises a scaffold region having a nucleic acid sequence at least 96% identical to SEQ ID NO: 24. In an aspect, a gRNA comprises a scaffold region having a nucleic acid sequence at least 97% identical to SEQ ID NO: 24. In an aspect, a gRNA comprises a scaffold region having a nucleic acid sequence at least 98% identical to SEQ ID NO: 24. In an aspect, a gRNA comprises a scaffold region having a nucleic acid sequence at least 99% identical to SEQ ID NO: 24. In an aspect, a gRNA comprises a scaffold region having a nucleic acid sequence 100% identical to SEQ ID NO: 24.

In an aspect, a gRNA comprises at least 20 nucleotides. In an aspect, a gRNA comprises at least 30 nucleotides. In an aspect, a gRNA comprises at least 40 nucleotides. In an aspect, a gRNA comprises at least 50 nucleotides. In an aspect, a gRNA comprises at least 60 nucleotides. In an aspect, a gRNA comprises at least 70 nucleotides. In an aspect, a gRNA comprises at least 80 nucleotides. In an aspect, a gRNA comprises at least 90 nucleotides. In an aspect, a gRNA comprises at least 100 nucleotides. In an aspect, a gRNA comprises at least 110 nucleotides. In an aspect, a gRNA comprises at least 120 nucleotides. In an aspect, a gRNA comprises at least 130 nucleotides. In an aspect, a gRNA comprises at least 140 nucleotides. In an aspect, a gRNA comprises at least 150 nucleotides. In an aspect, a gRNA comprises at least 175 nucleotides. In an aspect, a gRNA comprises at least 200 nucleotides. In an aspect, a gRNA comprises at least 250 nucleotides.

In an aspect, a gRNA comprises between 20 nucleotides and 500 nucleotides. In an aspect, a gRNA comprises between 20 nucleotides and 400 nucleotides. In an aspect, a gRNA comprises between 20 nucleotides and 300 nucleotides. In an aspect, a gRNA comprises between 20 nucleotides and 200 nucleotides. In an aspect, a gRNA comprises between 20 nucleotides and 150 nucleotides. In an aspect, a gRNA comprises between 20 nucleotides and 100 nucleotides. In an aspect, a gRNA comprises between 50 nucleotides and 250 nucleotides. In an aspect, a gRNA comprises between 50 nucleotides and 200 nucleotides. In an aspect, a gRNA comprises between 50 nucleotides and 150 nucleotides. In an aspect, a gRNA comprises between 75 nucleotides and 250 nucleotides. In an aspect, a gRNA comprises between 100 nucleotides and 250 nucleotides.

In an aspect, a gRNA is a prime editing gRNA (pegRNA). pegRNAs comprise a sequence to guide a protein to a target nucleic acid molecule on its 5′-end and a primer binding site region and a reverse transcriptase template sequence comprising a desired edit on its 3′-end.

In an aspect, a nucleic acid molecule comprises a homology arm. In an aspect, a nucleic acid molecule encodes a homology arm. As used herein, a “homology arm” refers to a nucleic acid molecule comprising a desired edit to be integrated into a target nucleic acid molecule, but is otherwise identical or complementary to the target nucleic acid molecule sequence. In an aspect, a homology arm is incorporated into a target nucleic acid molecule a reverse transcriptase. In an aspect, a homology arm is incorporated into a target nucleic acid molecule by a fusion protein comprising a reverse transcriptase. In an aspect, two homology arms are used to integrate a desired edit into a target nucleic acid molecule. In an aspect, one homology arm is used to integrate a desired edit into a target nucleic acid molecule.

In an aspect, a homology arm comprises DNA. In an aspect, a homology arm comprises RNA. In an aspect, a homology arm is single-stranded. In an aspect, a homology arm is double-stranded. In an aspect, a homology arm comprises at least 10 nucleotides. In an aspect, a homology arm comprises at least 20 nucleotides. In an aspect, a homology arm comprises at least 30 nucleotides. In an aspect, a homology arm comprises at least 40 nucleotides. In an aspect, a homology arm comprises at least 50 nucleotides. In an aspect, a homology arm comprises at least 60 nucleotides. In an aspect, a homology arm comprises at least 70 nucleotides. In an aspect, a homology arm comprises at least 75 nucleotides. In an aspect, a homology arm comprises at least 80 nucleotides. In an aspect, a homology arm comprises at least 90 nucleotides. In an aspect, a homology arm comprises at least 100 nucleotides. In an aspect, a homology arm comprises at least 250 nucleotides. In an aspect, a homology arm comprises at least 500 nucleotides. In an aspect, a homology arm comprises at least 750 nucleotides. In an aspect, a homology arm comprises at least 1000 nucleotides. In an aspect, a homology arm comprises at least 1500 nucleotides.

In an aspect, a homology arm comprises between 10 nucleotides and 2500 nucleotides. In an aspect, a homology arm comprises between 10 nucleotides and 1000 nucleotides. In an aspect, a homology arm comprises between 10 nucleotides and 500 nucleotides. In an aspect, a homology arm comprises between 10 nucleotides and 400 nucleotides. In an aspect, a homology arm comprises between 10 nucleotides and 300 nucleotides. In an aspect, a homology arm comprises between 10 nucleotides and 250 nucleotides. In an aspect, a homology arm comprises between 10 nucleotides and 125 nucleotides. In an aspect, a homology arm comprises between 10 nucleotides and 100 nucleotides. In an aspect, a homology arm comprises between 10 nucleotides and 75 nucleotides. In an aspect, a homology arm comprises between 10 nucleotides and 50 nucleotides. In an aspect, a homology arm comprises between 50 nucleotides and 500 nucleotides. In an aspect, a homology arm comprises between 50 nucleotides and 400 nucleotides. In an aspect, a homology arm comprises between 50 nucleotides and 300 nucleotides. In an aspect, a homology arm comprises between 50 nucleotides and 200 nucleotides. In an aspect, a homology arm comprises between 50 nucleotides and 150 nucleotides. In an aspect, a homology arm comprises between 50 nucleotides and 100 nucleotides. In an aspect, a homology arm comprises between 100 nucleotides and 1000 nucleotides. In an aspect, a homology arm comprises between 100 nucleotides and 500 nucleotides. In an aspect, a homology arm comprises between 250 nucleotides and 1000 nucleotides. In an aspect, a homology arm comprises between 250 nucleotides and 500 nucleotides. In an aspect, a homology arm comprises between 500 nucleotides and 2000 nucleotides. In an aspect, a homology arm comprises between 500 nucleotides and 1000 nucleotides.

When referring to a desired edit, a “control sequence” (also referred to herein as a “control version of a target nucleic acid molecule”) is used as a point of comparison. Any differences present in a homology arm as compared to the control sequence are to be considered the “desired edit” or “edit” for that homology arm. A control sequence refers to an unedited sequence. A control sequence can be naturally occurring or a transgenic or synthetically produced (e.g., man-made) sequence that does not occur in nature. For example, a sequence encoding GFP inserted into the genome of a yeast cell could serve a control sequence of a transgene that is to be edited. A control version of a target nucleic acid molecule refers to an unedited target nucleic acid molecule. A control version of a target nucleic acid molecule can be naturally occurring or a transgenic or synthetically produced (e.g., man-made) sequence that does not occur in nature.

In an aspect, a desired edit comprises a deletion of at least one nucleotide as compared to a control sequence. In an aspect, a desired edit comprises an insertion of at least one nucleotide as compared to a control sequence. In an aspect, a desired edit comprises a substitution of at least one nucleotide as compared to a control sequence. In an aspect, a desired edit comprises an inversion of at least two nucleotides as compared to a control sequence.

In an aspect, an edit comprises a deletion. In an aspect, at least 1 nucleotide is deleted from a target nucleic acid molecule as compared to a control version of the target nucleic acid molecule. In an aspect, at least 2 nucleotides are deleted from a target nucleic acid molecule as compared to a control version of the target nucleic acid molecule. In an aspect, at least 3 nucleotides are deleted from a target nucleic acid molecule as compared to a control version of the target nucleic acid molecule. In an aspect, at least 4 nucleotides are deleted from a target nucleic acid molecule as compared to a control version of the target nucleic acid molecule. In an aspect, at least 5 nucleotides are deleted from a target nucleic acid molecule as compared to a control version of the target nucleic acid molecule. In an aspect, at least 10 nucleotides are deleted from a target nucleic acid molecule as compared to a control version of the target nucleic acid molecule. In an aspect, at least 15 nucleotides are deleted from a target nucleic acid molecule as compared to a control version of the target nucleic acid molecule. In an aspect, at least 20 nucleotides are deleted from a target nucleic acid molecule as compared to a control version of the target nucleic acid molecule. In an aspect, at least 25 nucleotides are deleted from a target nucleic acid molecule as compared to a control version of the target nucleic acid molecule. In an aspect, at least 30 nucleotides are deleted from a target nucleic acid molecule as compared to a control version of the target nucleic acid molecule. In an aspect, at least 40 nucleotides are deleted from a target nucleic acid molecule as compared to a control version of the target nucleic acid molecule. In an aspect, at least 50 nucleotides are deleted from a target nucleic acid molecule as compared to a control version of the target nucleic acid molecule. In an aspect, at least 75 nucleotides are deleted from a target nucleic acid molecule as compared to a control version of the target nucleic acid molecule. In an aspect, at least 100 nucleotides are deleted from a target nucleic acid molecule as compared to a control version of the target nucleic acid molecule. In an aspect, at least 250 nucleotides are deleted from a target nucleic acid molecule as compared to a control version of the target nucleic acid molecule.

In an aspect, at least 500 nucleotides are deleted from a target nucleic acid molecule as compared to a control version of the target nucleic acid molecule.

In an aspect, an edit comprises an insertion. In an aspect, at least 1 nucleotide is inserted into a target nucleic acid molecule as compared to a control version of the target nucleic acid molecule. In an aspect, at least 2 nucleotides are inserted into a target nucleic acid molecule as compared to a control version of the target nucleic acid molecule. In an aspect, at least 3 nucleotides are inserted into a target nucleic acid molecule as compared to a control version of the target nucleic acid molecule. In an aspect, at least 4 nucleotides are inserted into a target nucleic acid molecule as compared to a control version of the target nucleic acid molecule. In an aspect, at least 5 nucleotides are inserted into a target nucleic acid molecule as compared to a control version of the target nucleic acid molecule. In an aspect, at least 10 nucleotides are inserted into a target nucleic acid molecule as compared to a control version of the target nucleic acid molecule. In an aspect, at least 15 nucleotides are inserted into a target nucleic acid molecule as compared to a control version of the target nucleic acid molecule. In an aspect, at least 20 nucleotides are inserted into a target nucleic acid molecule as compared to a control version of the target nucleic acid molecule. In an aspect, at least 25 nucleotides are inserted into a target nucleic acid molecule as compared to a control version of the target nucleic acid molecule. In an aspect, at least 30 nucleotides are inserted into a target nucleic acid molecule as compared to a control version of the target nucleic acid molecule. In an aspect, at least 40 nucleotides are inserted into a target nucleic acid molecule as compared to a control version of the target nucleic acid molecule. In an aspect, at least 50 nucleotides are inserted into a target nucleic acid molecule as compared to a control version of the target nucleic acid molecule. In an aspect, at least 75 nucleotides are inserted into a target nucleic acid molecule as compared to a control version of the target nucleic acid molecule. In an aspect, at least 100 nucleotides are inserted into a target nucleic acid molecule as compared to a control version of the target nucleic acid molecule. In an aspect, at least 250 nucleotides are inserted into a target nucleic acid molecule as compared to a control version of the target nucleic acid molecule. In an aspect, at least 500 nucleotides are inserted into a target nucleic acid molecule as compared to a control version of the target nucleic acid molecule.

In an aspect, an edit comprises a substitution. In an aspect, an edit comprises a substitution of a single nucleotide in a target nucleic acid molecule as compared to a control version of the target nucleic acid molecule. In an aspect, an edit comprises a substitution of at least 2 nucleotides in a target nucleic acid molecule as compared to a control version of the target nucleic acid molecule. When more than one nucleotide is substituted in a nucleic acid molecule, the substitutions do not need to be adjacent to each other. Two or more nucleotide substitutions can be separated by non-edited nucleotides. In an aspect, an edit comprises a substitution of at least 3 nucleotides in a target nucleic acid molecule as compared to a control version of the target nucleic acid molecule. In an aspect, an edit comprises a substitution of at least 4 nucleotides in a target nucleic acid molecule as compared to a control version of the target nucleic acid molecule. In an aspect, an edit comprises a substitution of at least 5 nucleotides in a target nucleic acid molecule as compared to a control version of the target nucleic acid molecule. In an aspect, an edit comprises a substitution of at least 6 nucleotides in a target nucleic acid molecule as compared to a control version of the target nucleic acid molecule. In an aspect, an edit comprises a substitution of at least 7 nucleotides in a target nucleic acid molecule as compared to a control version of the target nucleic acid molecule. In an aspect, an edit comprises a substitution of at least 8 nucleotides in a target nucleic acid molecule as compared to a control version of the target nucleic acid molecule. In an aspect, an edit comprises a substitution of at least 9 nucleotides in a target nucleic acid molecule as compared to a control version of the target nucleic acid molecule. In an aspect, an edit comprises a substitution of at least 10 nucleotides in a target nucleic acid molecule as compared to a control version of the target nucleic acid molecule. In an aspect, an edit comprises a substitution of at least 15 nucleotides in a target nucleic acid molecule as compared to a control version of the target nucleic acid molecule. In an aspect, an edit comprises a substitution of at least 20 nucleotides in a target nucleic acid molecule as compared to a control version of the target nucleic acid molecule. In an aspect, an edit comprises a substitution of at least 30 nucleotides in a target nucleic acid molecule as compared to a control version of the target nucleic acid molecule. In an aspect, an edit comprises a substitution of at least 40 nucleotides in a target nucleic acid molecule as compared to a control version of the target nucleic acid molecule. In an aspect, an edit comprises a substitution of at least 50 nucleotides in a target nucleic acid molecule as compared to a control version of the target nucleic acid molecule.

In an aspect, an edit comprises an inversion. In an aspect, an edit comprises an inversion of at least 2 nucleotides in a target nucleic acid molecule as compared to a control version of the target nucleic acid molecule. In an aspect, an edit comprises an inversion of at least 3 nucleotides in a target nucleic acid molecule as compared to a control version of the target nucleic acid molecule. In an aspect, an edit comprises an inversion of at least 4 nucleotides in a target nucleic acid molecule as compared to a control version of the target nucleic acid molecule. In an aspect, an edit comprises an inversion of at least 5 nucleotides in a target nucleic acid molecule as compared to a control version of the target nucleic acid molecule. In an aspect, an edit comprises an inversion of at least 10 nucleotides in a target nucleic acid molecule as compared to a control version of the target nucleic acid molecule. In an aspect, an edit comprises an inversion of at least 20 nucleotides in a target nucleic acid molecule as compared to a control version of the target nucleic acid molecule. In an aspect, an edit comprises an inversion of at least 30 nucleotides in a target nucleic acid molecule as compared to a control version of the target nucleic acid molecule. In an aspect, an edit comprises an inversion of at least 40 nucleotides in a target nucleic acid molecule as compared to a control version of the target nucleic acid molecule. In an aspect, an edit comprises an inversion of at least 50 nucleotides in a target nucleic acid molecule as compared to a control version of the target nucleic acid molecule. In an aspect, an edit comprises an inversion of at least 75 nucleotides in a target nucleic acid molecule as compared to a control version of the target nucleic acid molecule. In an aspect, an edit comprises an inversion of at least 100 nucleotides in a target nucleic acid molecule as compared to a control version of the target nucleic acid molecule. In an aspect, an edit comprises an inversion of at least 150 nucleotides in a target nucleic acid molecule as compared to a control version of the target nucleic acid molecule. In an aspect, an edit comprises an inversion of at least 200 nucleotides in a target nucleic acid molecule as compared to a control version of the target nucleic acid molecule. In an aspect, an edit comprises an inversion of at least 500 nucleotides in a target nucleic acid molecule as compared to a control version of the target nucleic acid molecule. In an aspect, an edit comprises an inversion of at least 1000 nucleotides in a target nucleic acid molecule as compared to a control version of the target nucleic acid molecule.

In an aspect, an edit comprises at least one deletion and at least one insertion. In an aspect, an edit comprises at least one deletion and at least one substitution. In an aspect, an edit comprises at least one deletion and at least one inversion. In an aspect, an edit comprises at least one insertion and at least one substitution. In an aspect, an edit comprises at least one insertion and at least one inversion. In an aspect, an edit comprises at least one substitution and at least one inversion.

In an aspect, an edit comprises at least one deletion, at least one insertion, and at least one substitution. In an aspect, an edit comprises at least one deletion, at least one insertion, and at least one inversion. In an aspect, an edit comprises at least one insertion, at least one substitution, and at least one inversion.

In an aspect, an edit comprises at least one deletion, at least one insertion, at least one substitution, and at least one inversion.

In an aspect, an edit comprises the introduction of a premature stop codon into a nucleic acid sequence encoding a protein. In an aspect, an edit results in a null mutation in the target nucleic acid molecule.

In an aspect, an edit comprises one or more mutation types selected from the group consisting of a nonsense edit, a missense edit, a frameshift edit, a splice-site edit, and any combinations thereof. As used herein, a “nonsense edit” refers to an edit to a nucleic acid sequence that introduces a premature stop codon to an amino acid sequence encoded by the nucleic acid sequence. As used herein, a “missense edit” refers to an edit to a nucleic acid sequence that causes a substitution within the amino acid sequence encoded by the nucleic acid sequence. As used herein, a “frameshift edit” refers to an insertion or deletion to a nucleic acid sequence that shifts the frame for translating the nucleic acid sequence to an amino acid sequence. A “splice-site edit” refers to an edit in a nucleic acid sequence that causes an intron to be retained for protein translation, or, alternatively, for an exon to be excluded from protein translation. Splice-site edits can cause nonsense, missense, or frameshift edits.

Edits in coding regions of genes (e.g., exonic edits) can result in a truncated protein or polypeptide when a mutated messenger RNA (mRNA) is translated into a protein or polypeptide. In an aspect, this disclosure provides an edit that results in the truncation of a protein or polypeptide. As used herein, a “truncated” protein or polypeptide comprises at least one fewer amino acid as compared to an endogenous control protein or polypeptide. For example, if endogenous Protein A comprises 100 amino acids, a truncated version of Protein A can comprise between 1 and 99 amino acids.

Without being limited by any scientific theory, one way to cause a protein or polypeptide truncation is by the introduction of a premature stop codon in an mRNA transcript of an endogenous gene. In an aspect, this disclosure provides an edit that results in a premature stop codon in an mRNA transcript of an endogenous gene. As used herein, a “stop codon” refers to a nucleotide triplet within an mRNA transcript that signals a termination of protein translation. A “premature stop codon” refers to a stop codon positioned earlier (e.g., on the 5′-side) than the normal stop codon position in an endogenous mRNA transcript. Without being limiting, several stop codons are known in the art, including “UAG,” “UAA,” “UGA,” “TAG,” “TAA,” and “TGA.”

As used herein, a “null edit” refers to an edit that confers a complete loss-of-function for a protein encoded by a gene comprising the edit, or, alternatively, an edit that confers a complete loss-of-function for a small RNA encoded by a genomic locus. A null edit can cause lack of mRNA transcript production, a lack of small RNA transcript production, a lack of protein function, or a combination thereof.

When a protein or nucleoprotein complex “edits” a target nucleic acid molecule, the protein or nucleoprotein complex causes at least one deletion, insertion, substitution, or inversion in the target nucleic acid molecule sequence as compared to a control version of the target nucleic acid molecule sequence. In an aspect, a nucleoprotein complex edits a target nucleic acid molecule within a cell.

In an aspect, the substitution of a single nucleotide comprises a transition. A transition substitution is a substitution of one purine for another (e.g., adenine for guanine or vice versa) or a substitution of one pyrimidine for another (e.g., cytosine for thymine or vice versa).

In an aspect, the substitution of a single nucleotide comprises a transversion. A transversion substitution is a substitution of one purine for a pyrimidine or vice versa (e.g., adenine for cytosine or vice versa; adenine for thymine or vice versa; guanine for cytosine or vice versa; guanine for thymine or vice versa).

In an aspect, an edit is positioned within an exon of a target nucleic acid molecule. In an aspect, an edit is positioned within an intron of a target nucleic acid molecule. In an aspect, an edit is positioned within a 5′-untranslated region (UTR) of a target nucleic acid molecule. In an aspect, an edit is positioned within a 3′-UTR of a target nucleic acid molecule. In an aspect, an edit is positioned within a non-coding region of a target nucleic acid molecule. In an aspect, an edit is positioned with a coding region of a target nucleic acid molecule. A coding region of a target nucleic acid molecule can encode a protein or a non-coding RNA. In an aspect, an edit is positioned within a gene in a target nucleic acid molecule. In an aspect, an edit is positioned within a promoter. In an aspect, an edit is positioned within a transcription terminator. In an aspect, an edit is positioned within a polyadenylation site.

As used herein, a “target nucleic acid molecule” refers to any nucleic acid molecule comprising a nucleic acid sequence that is desired to be edited. In an aspect, a target nucleic acid molecule is positioned within a genome of a cell. In an aspect, a target nucleic acid molecule is positioned within a nuclear genome. In an aspect, a target nucleic acid molecule is positioned within a mitochondrial genome. In an aspect, a target nucleic acid molecule is positioned within a chloroplast genome. In an aspect, a target nucleic acid molecule is positioned within a plasmid. In an aspect, a target nucleic acid molecule is double-stranded. In an aspect, a target nucleic acid molecule is a DNA molecule.

In an aspect, a target nucleic acid molecule comprises a gene. In an aspect, a target nucleic acid molecule comprises a promoter. In an aspect, a target nucleic acid molecule comprises a protein-coding sequence. In an aspect, a target nucleic acid molecule comprises a sequence encoding a non-coding RNA molecule. Non-coding RNAs are RNA molecules that are not translated into proteins. Non-limiting examples of non-coding RNA molecules include microRNAs (miRNAs), small interfering RNAs (siRNAs), Piwi-interacting RNAs (piRNAs), small nucleolar RNAs (snoRNAs), extracellular RNAs (exRNAs), gRNAs, and others.

In an aspect, a target nucleic acid molecule comprises at least one exon. In an aspect, a target nucleic acid molecule comprises at least one intron. In an aspect, a target nucleic acid molecule comprises an untranslated region (e.g., 5′-UTR, 3′-UTR).

In an aspect, a target nucleic acid molecule encodes a reporter gene. As used herein, a “reporter gene” refers to any gene that can be used to assay for the transcriptional activity of an operably linked promoter. Reporter gene activity can be detected, without being limiting, by MRI, PET, visualization of bioluminescence or fluorescence, color change, and whether a cell is capable of growing on a certain media. Without being limiting, editing a reporter gene can be used to determine the editing efficiency and/or effectiveness of a fusion protein or of a MAD2019-H848A variant polypeptide. See, for example, Example 1.

In an aspect, a reporter gene encodes a fluorescent molecule. As used herein, a “fluorescent molecule” refers to a molecule that can re-emit light upon excitation. Fluorescent molecules are also referred to as fluorophores in the art. In an aspect, a fluorescent molecule is GREEN FLUORSCENT PROTEIN (GFP). In an aspect, a fluorescent molecule is RED FLUORESCENT PROTEIN (RFP). In an aspect, a fluorescent molecule is YELLOW FLUORESCENT PROTEIN (YFP). In an aspect, a fluorescent molecule is CYAN FLUORESCENT PROTEIN (CFP). In an aspect, a fluorescent molecule is selected from the group consisting of mCherry, mOrange, mRaspberry, mKO, TagRFP, mKate, mRuby, FusionRed, mScarlet, and DsRed-Express.

In an aspect, a reporter gene encodes a bioluminescent molecule. In an aspect, a bioluminescent molecule is luciferase.

In an aspect, a reporter gene encodes β-glucuronidase (GUS).

In an aspect, this disclosure provides a cell comprising any polypeptide, fusion protein, DNA molecule, or RNA molecule provided herein. In an aspect, this disclosure provides a cell comprising any nucleoprotein complex provided herein. In an aspect, this disclosure provides a cell comprising any MAD2019-H848A polypeptide variant provided herein. In an aspect, this disclosure provides a cell comprising any fusion protein provided herein. In an aspect, this disclosure provides a cell comprising any guide provided herein. In an aspect, this disclosure provides a cell comprising any homology arm provided herein. In an aspect, this disclosure provides a cell comprising any reverse transcriptase provided herein.

In an aspect, a cell is a prokaryotic cell. In an aspect, a prokaryotic cell is a bacteria cell. In an aspect, a prokaryotic cell is an archaea cell. In in aspect, a prokaryotic cell is an Escherichia coli cell.

In an aspect, a cell is a eukaryotic cell.

In an aspect, this disclosure provides a eukaryotic cell comprising any polypeptide, fusion protein, DNA molecule, or RNA molecule provided herein. In an aspect, this disclosure provides a eukaryotic cell comprising any nucleoprotein complex provided herein. In an aspect, this disclosure provides a eukaryotic cell comprising any MAD2019-H848A polypeptide variant provided herein. In an aspect, this disclosure provides a eukaryotic cell comprising any fusion protein provided herein.

In an aspect, a eukaryotic cell is an animal cell. In an aspect, a eukaryotic cell is selected from the group consisting of a fish cell, a bird cell, a reptile cell, an amphibian cell, an insect cell, an arachnid cell, a flatworm cell, an annelid cell, and a crustacean cell. In an aspect, a eukaryotic cell is a mammal cell. In an aspect, a mammal cell is a primate cell. In an aspect, a eukaryotic cell is a human cell. In an aspect, a mammal cell is selected from the group consisting of a cat cell, a dog cell, a lagomorph cell, a rodent cell, an ungulate cell, a marsupial cell, and a bat cell.

In an aspect, a eukaryotic cell is an in vivo cell. In an aspect, a eukaryotic cell is an ex vivo cell.

In an aspect, a eukaryotic cell is not a human cell. In an aspect, a eukaryotic cell is a non-human mammalian cell. In an aspect, a eukaryotic cell is a non-human animal cell. In an aspect, a human cell is an ex vivo cell.

In an aspect, human cell is an induced pluripotent stem cell. In an aspect, a human cell is an HEK293T cell.

In an aspect, a human cell is selected from the group consisting of a bone cell, a ligament cell, a tendon cell, a muscle cell, a tongue cell, a lip cell, a salivary gland cell, a pharynx cell, an esophagus cell, a stomach cell, a small intestine cell, a large intestine cell, a rectum cell, a liver cell, a gallbladder cell, a mesentery cell, a pancreas cell, a nasal cell, a pharynx cell, a larynx cell, a trachea cell, a bronchi cell, a bronchiole cell, a lung cell, a kidney cell, a ureter cell, a bladder cell, a urethra cell, a reproductive system cell, a pituitary gland cell, a pineal gland cell, a thyroid gland cell, a parathyroid gland cell, an adrenal gland cell, a pancreas cell, a heart cell, an artery cell, a vein cell, a capillary cell, a lymphatic vessel cell, a lymph node cell, a bone marrow cell, a thymus cell, a spleen cell, a tonsil cell, an interstitum cell, a brain cell, a spinal chord cell, a nerve cell, an eye cell, and an ear cell.

In an aspect, a eukaryotic cell is a fungal cell. In an aspect, a eukaryotic cell is a yeast cell. In an aspect, a eukaryotic cell is a Schizosaccharomyces pombe cell. In an aspect, a eukaryotic cell is a Saccharomyces cerevisiae cell.

In an aspect, a eukaryotic cell is a plant cell.

In an aspect, this disclosure provides a fusion protein comprising a MAD2019-H848A variant polypeptide. In an aspect, a fusion protein comprises a MAD2019-H848A variant polypeptide and a reverse transcriptase. In an aspect, this disclosure provides a fusion protein comprising a Tf1 reverse transcriptase comprising an amino acid sequence at least 80% identical or similar to SEQ ID NO: 12. In an aspect, this disclosure provides a fusion protein comprising a Tf1 reverse transcriptase comprising an amino acid sequence at least 80% identical or similar to SEQ ID NO: 13, where the amino acid sequence comprises an asparagine at position 364 as compared to SEQ ID NO: 13. In an aspect, this disclosure provides a fusion protein comprising a Tf1 reverse transcriptase comprising the amino acid sequence of SEQ ID NO: 13.

As used herein, a “fusion protein” refers to a protein created by joining two or more polypeptide (or protein) amino acid sequences together. In an aspect, a fusion protein is encoded by a single nucleic acid molecule. In an aspect, a fusion protein comprises a nuclease and a reverse transcriptase. In an aspect, a fusion protein comprises a nickase and a reverse transcriptase.

In an aspect, a fusion protein comprises a nickase comprising SEQ ID NO: 1 and an HIV-1 reverse transcriptase. In an aspect, a fusion protein comprises a nickase comprising SEQ ID NO: 1 and an M-MLV reverse transcriptase. In an aspect, a fusion protein comprises a nickase comprising SEQ ID NO: 1 and an AMV reverse transcriptase. In an aspect, a fusion protein comprises a nickase comprising SEQ ID NO: 16 and an HIV-1 reverse transcriptase. In an aspect, a fusion protein comprises a nickase comprising SEQ ID NO: 16 and an M-MLV reverse transcriptase. In an aspect, a fusion protein comprises a nickase comprising SEQ ID NO: 16 and an AMV reverse transcriptase.

In an aspect, a fusion protein comprises a MAD2019-H848A variant polypeptide and a Tf1 reverse transcriptase. In an aspect, a fusion protein comprises a MAD2019-H848A variant polypeptide and a Tf1-D364N reverse transcriptase. In an aspect, a fusion protein comprises a MAD2019-H848A variant polypeptide and an HIV-1 reverse transcriptase. In an aspect, a fusion protein comprises a MAD2019-H848A variant polypeptide and an M-MLV reverse transcriptase. In an aspect, a fusion protein comprises a MAD2019-H848A variant polypeptide and an AMV reverse transcriptase.

In an aspect, a fusion protein comprises an amino acid sequence selected from the group consisting of SEQ ID NOs: 20 to 23.

In an aspect, a fusion protein comprises a linker amino acid sequence. As used herein, a “linker amino acid sequence” refers to amino acid residues placed between the two or more polypeptide sequences that comprise a fusion protein. It will be appreciated that a linker amino acid sequence has no enzymatic activity on its own. In an aspect, a linker amino acid sequence is positioned between a nickase and a reverse transcriptase. In an aspect, a linker amino acid sequence is positioned between a MAD2019-H848A variant polypeptide and a reverse transcriptase. In an aspect, a linker amino acid sequence is positioned between a MAD2019-H848A variant polypeptide and a Tf1 reverse transcriptase. In an aspect, a linker amino acid sequence is positioned between a MAD2019-H848A variant polypeptide and a Tf1-D364N reverse transcriptase. In an aspect, a linker amino acid sequence is positioned between a MAD2019-H848A variant polypeptide and an HIV-1 reverse transcriptase. In an aspect, a linker amino acid sequence is positioned between a MAD2019-H848A variant polypeptide and an M-MLV reverse transcriptase. In an aspect, a linker amino acid sequence is positioned between a MAD2019-H848A variant polypeptide and an AMV reverse transcriptase.

In an aspect, a linker amino acid sequence is positioned between an NLS and a nickase. In an aspect, a linker amino acid sequence is positioned between an NLS and a reverse transcriptase. In an aspect, a linker amino acid sequence is positioned between an NLS and a MAD2019-H848A variant polypeptide. In an aspect, a linker amino acid sequence is positioned between an NLS and a Tf1 reverse transcriptase. In an aspect, a linker amino acid sequence is positioned between an NLS and a Tf1-D364N reverse transcriptase. In an aspect, a linker amino acid sequence is positioned between an NLS and an HIV-1 reverse transcriptase. In an aspect, a linker amino acid sequence is positioned between an NLS and an M-MLV reverse transcriptase. In an aspect, a linker amino acid sequence is positioned between an NLS and an AMV reverse transcriptase

In an aspect, a linker amino acid sequence comprises at least 1 amino acid residue. In an aspect, a linker amino acid sequence comprises at least 2 amino acid residues. In an aspect, a linker amino acid sequence comprises at least 3 amino acid residues. In an aspect, a linker amino acid sequence comprises at least 4 amino acid residues. In an aspect, a linker amino acid sequence comprises at least 5 amino acid residues. In an aspect, a linker amino acid sequence comprises at least 6 amino acid residues. In an aspect, a linker amino acid sequence comprises at least 7 amino acid residues. In an aspect, a linker amino acid sequence comprises at least 8 amino acid residues. In an aspect, a linker amino acid sequence comprises at least 9 amino acid residues. In an aspect, a linker amino acid sequence comprises at least 10 amino acid residues. In an aspect, a linker amino acid sequence comprises at least 15 amino acid residues. In an aspect, a linker amino acid sequence comprises at least 20 amino acid residues. In an aspect, a linker amino acid sequence comprises at least 25 amino acid residues. In an aspect, a linker amino acid sequence comprises at least 30 amino acid residues. In an aspect, a linker amino acid sequence comprises at least 35 amino acid residues. In an aspect, a linker amino acid sequence comprises at least 40 amino acid residues. In an aspect, a linker amino acid sequence comprises at least 45 amino acid residues. In an aspect, a linker amino acid sequence comprises at least 50 amino acid residues. In an aspect, a linker amino acid sequence comprises at least 60 amino acid residues. In an aspect, a linker amino acid sequence comprises at least 70 amino acid residues.

In an aspect, a linker amino acid sequence comprises between 1 amino acid residue and 75 amino acid residues. In an aspect, a linker amino acid sequence comprises between 1 amino acid residue and 50 amino acid residues. In an aspect, a linker amino acid sequence comprises between 1 amino acid residue and 40 amino acid residues. In an aspect, a linker amino acid sequence comprises between 1 amino acid residue and 30 amino acid residues. In an aspect, a linker amino acid sequence comprises between 1 amino acid residue and 20 amino acid residues. In an aspect, a linker amino acid sequence comprises between 1 amino acid residue and 10 amino acid residues. In an aspect, a linker amino acid sequence comprises between 10 amino acid residues and 50 amino acid residues. In an aspect, a linker amino acid sequence comprises between 10 amino acid residues and 40 amino acid residues. In an aspect, a linker amino acid sequence comprises between 20 amino acid residues and 50 amino acid residues. In an aspect, a linker amino acid sequence comprises between 20 amino acid residues and 40 amino acid residues.

As used herein, “reverse transcriptase” reverse to any enzyme than can generate complementary DNA (cDNA) from an RNA template. Reverse transcriptases are classified under section 2.7.7.49 by the Enzyme Commission and the CAS Registry Number® 9068-38-6. Non-limiting examples of reverse transcriptase amino acid sequences are provided as SEQ ID NOs: 12 and 13 (which are both Tf1 reverse transcriptases).

In an aspect, a reverse transcriptase is a Tf1 reverse transcriptase. In an aspect, a reverse transcriptase is derived from a Tf1 reverse transcriptase. In an aspect, a reverse transcriptase is an human immunodeficiency virus-1 (HIV-1) reverse transcriptase. In an aspect, a reverse transcriptase is derived from an HIV-1 reverse transcriptase. In an aspect, a reverse transcriptase is a Moloney murine leukemia virus (M-MLV) reverse transcriptase. In an aspect, a reverse transcriptase is derived from an M-MLV reverse transcriptase. In an aspect, a reverse transcriptase is an avian myeloblastosis virus (AMV) reverse transcriptase. In an aspect, a reverse transcriptase is derived from an AMV reverse transcriptase. In an aspect, a reverse transcriptase is selected from the group consisting of an HIV-1 reverse transcriptase, an M-MLV reverse transcriptase, and an AMV reverse transcriptase. In an aspect, a reverse transcriptase is derived from a reverse transcriptase selected from the group consisting of an HIV-1 reverse transcriptase, an M-MLV reverse transcriptase, and an AMV reverse transcriptase.

In an aspect, a reverse transcriptase comprises an amino acid sequence at least 80% identical or similar to SEQ ID NO: 12. In an aspect, a reverse transcriptase comprises an amino acid sequence at least 85% identical or similar to SEQ ID NO: 12. In an aspect, a reverse transcriptase comprises an amino acid sequence at least 90% identical or similar to SEQ ID NO: 12. In an aspect, a reverse transcriptase comprises an amino acid sequence at least 91% identical or similar to SEQ ID NO: 12. In an aspect, a reverse transcriptase comprises an amino acid sequence at least 92% identical or similar to SEQ ID NO: 12. In an aspect, a reverse transcriptase comprises an amino acid sequence at least 93% identical or similar to SEQ ID NO: 12. In an aspect, a reverse transcriptase comprises an amino acid sequence at least 94% identical or similar to SEQ ID NO: 12. In an aspect, a reverse transcriptase comprises an amino acid sequence at least 95% identical or similar to SEQ ID NO: 12. In an aspect, a reverse transcriptase comprises an amino acid sequence at least 96% identical or similar to SEQ ID NO: 12. In an aspect, a reverse transcriptase comprises an amino acid sequence at least 97% identical or similar to SEQ ID NO: 12. In an aspect, a reverse transcriptase comprises an amino acid sequence at least 98% identical or similar to SEQ ID NO: 12. In an aspect, a reverse transcriptase comprises an amino acid sequence at least 99% identical or similar to SEQ ID NO: 12. In an aspect, a reverse transcriptase comprises an amino acid sequence 100% identical or similar to SEQ ID NO: 12.

In an aspect, a reverse transcriptase comprises an amino acid sequence at least 80% identical or similar to SEQ ID NO: 13, wherein the amino acid sequence comprises an asparagine at position 364 as compared to SEQ ID NO: 13. In an aspect, a reverse transcriptase comprises an amino acid sequence at least 85% identical or similar to SEQ ID NO: 13, wherein the amino acid sequence comprises an asparagine at position 364 as compared to SEQ ID NO: 13. In an aspect, a reverse transcriptase comprises an amino acid sequence at least 90% identical or similar to SEQ ID NO: 13, wherein the amino acid sequence comprises an asparagine at position 364 as compared to SEQ ID NO: 13. In an aspect, a reverse transcriptase comprises an amino acid sequence at least 91% identical or similar to SEQ ID NO: 13, wherein the amino acid sequence comprises an asparagine at position 364 as compared to SEQ ID NO: 13. In an aspect, a reverse transcriptase comprises an amino acid sequence at least 92% identical or similar to SEQ ID NO: 13, wherein the amino acid sequence comprises an asparagine at position 364 as compared to SEQ ID NO: 13. In an aspect, a reverse transcriptase comprises an amino acid sequence at least 93% identical or similar to SEQ ID NO: 13, wherein the amino acid sequence comprises an asparagine at position 364 as compared to SEQ ID NO: 13. In an aspect, a reverse transcriptase comprises an amino acid sequence at least 94% identical or similar to SEQ ID NO: 13, wherein the amino acid sequence comprises an asparagine at position 364 as compared to SEQ ID NO: 13. In an aspect, a reverse transcriptase comprises an amino acid sequence at least 95% identical or similar to SEQ ID NO: 13, wherein the amino acid sequence comprises an asparagine at position 364 as compared to SEQ ID NO: 13. In an aspect, a reverse transcriptase comprises an amino acid sequence at least 96% identical or similar to SEQ ID NO: 13, wherein the amino acid sequence comprises an asparagine at position 364 as compared to SEQ ID NO: 13. In an aspect, a reverse transcriptase comprises an amino acid sequence at least 97% identical or similar to SEQ ID NO: 13, wherein the amino acid sequence comprises an asparagine at position 364 as compared to SEQ ID NO: 13. In an aspect, a reverse transcriptase comprises an amino acid sequence at least 98% identical or similar to SEQ ID NO: 13, wherein the amino acid sequence comprises an asparagine at position 364 as compared to SEQ ID NO: 13. In an aspect, a reverse transcriptase comprises an amino acid sequence at least 99% identical or similar to SEQ ID NO: 13, wherein the amino acid sequence comprises an asparagine at position 364 as compared to SEQ ID NO: 13. In an aspect, a reverse transcriptase comprises an amino acid sequence 100% identical or similar to SEQ ID NO: 13.

In an aspect, a fusion protein comprising a Tf1 reverse transcriptase further comprises a nickase. In an aspect, a fusion protein comprising a Tf1 reverse transcriptase further comprises a CRISPR-Cas nickase. In an aspect, a fusion protein comprising a Tf1 reverse transcriptase further comprises a type I CRISPR-Cas nickase. In an aspect, a fusion protein comprising a Tf1 reverse transcriptase further comprises a type II CRISPR-Cas nickase. In an aspect, a fusion protein comprising a Tf1 reverse transcriptase further comprises a type III CRISPR-Cas nickase. In an aspect, a fusion protein comprising a Tf1 reverse transcriptase further comprises a type IV CRISPR-Cas nickase. In an aspect, a fusion protein comprising a Tf1 reverse transcriptase further comprises a type V CRISPR-Cas nickase. In an aspect, a fusion protein comprising a Tf1 reverse transcriptase further comprises a type VI CRISPR-Cas nickase. In an aspect, a fusion protein comprising a Tf1 reverse transcriptase further comprises a Cas9 nickase. In an aspect, a fusion protein comprising a Tf1 reverse transcriptase further comprises a MAD2019 nickase. In an aspect, a fusion protein comprising a Tf1 reverse transcriptase further comprises a MAD2019-H848A polypeptide. In an aspect, a fusion protein comprising a Tf1 reverse transcriptase further comprises a MAD2019-H848A variant polypeptide.

In an aspect, a CRISPR-Cas nickase is a type I CRISPR-Cas-derived nickase. In an aspect, a CRISPR-Cas nickase is a type II CRISPR-Cas-derived nickase. In an aspect, a CRISPR-Cas nickase is a type III CRISPR-Cas-derived nickase. In an aspect, a CRISPR-Cas nickase is a type IV CRISPR-Cas-derived nickase. In an aspect, a CRISPR-Cas nickase is a type V CRISPR-Cas-derived nickase. In an aspect, a CRISPR-Cas nickase is a type VI CRISPR-Cas-derived nickase. In an aspect, a CRISPR-Cas nickase is a Cas9 nickase. In an aspect, a CRISPR-Cas nickase is a Cas9-derived nickase. In an aspect, a CRISPR-Cas nickase is a MAD2019-derived nickase. In an aspect, a CRISPR-Cas nickase is a MAD2019-H848A polypeptide. In an aspect, a CRISPR-Cas nickase is a MAD2019-H848A variant polypeptide.

CRISPR-Cas (Clustered Regularly Interspaced Short Palindromic Repeats-CRISPR associated proteins) enzymes are nucleases that use guides to recognize and cleave specific DNA targets. Without being limited by any scientific theory, nickases can be derived from nucleases (e.g., CRISPR-Cas enzymes) by mutating or editing the nucleases. In an aspect, a nickase is derived from a CRISPR-Cas enzyme.

Non-limiting examples of type I CRISPR-Cas enzymes include Cas3, Cas5, Cas8a, Cas8b, Cas8c, Cas10d, Cse1, Cse2, Csy1, Csy2, Csy3, and GSU0054. Non-limiting examples of type II CRISPR-Cas enzymes include Cas4, Cas9, and Csn2. Non-limiting examples of type III CRISPR-Cas enzymes include Cas10, Csm2, Cmr5, Csx10, and Csx11. A non-limiting example of type IV CRISPR-Cas enzymes is Csf1. Non-limiting examples of type V CRISPR-Cas enzymes include Cas12, Cas12a (also known as Cpf1), Cas12b (also known as C2c1), Cas12c (also known as C2c3), Cas12d (also known as CasY), Cas12e (also known as CasX), Cas12f (also known as Cas14 or C2c10), Cas12g, Cas12h, Cas12i, Cas12k (also known as C2c5), C2c4, C2c8, and C2c9. Non-limiting examples of type VI CRISPR-Cas enzymes include Cas13, Cas13a (also known as C2c2), Cas13b, Cas13c, and Cas13d.

In an aspect, this disclosure provides a method of providing a MAD2019-H848A variant polypeptide to a cell, the method comprising: (a) obtaining a cell; and (b) providing the cell with a MAD2019-H848A variant polypeptide or a nucleic acid molecule encoding the MAD2019-H848A variant polypeptide. In an aspect, this disclosure provides a method of providing a fusion protein to a cell, the method comprising: (a) obtaining a cell; and (b) providing the cell with a fusion protein or a nucleic acid molecule encoding the fusion protein. In an aspect, the method further comprises transfecting the cell with a nucleic acid molecule encoding a guide. In an aspect, the method further comprises transfecting the cell with a nucleic acid molecule encoding a homology arm. In an aspect, the method further comprises transfecting the cell with a nucleic acid molecule encoding a guide and a homology arm. In an aspect, the method further comprises transfecting the cell with a nucleic acid molecule encoding (a) a guide; (b) a homology arm; or (c) a guide and a homology arm.

In an aspect, a nucleic acid molecule provided herein is stably integrated into a genome in a cell. In an aspect, a nucleic acid molecule provided herein is transiently introduced into a cell. In an aspect, a nucleic acid molecule provided herein is positioned within a plasmid. As used herein, a “plasmid” refers to a circular, double-stranded DNA molecule. In an aspect, a plasmid comprises an origin of replication. In an aspect, a plasmid comprises a selectable marker gene.

Methods involving transient transformation or stable integration of any nucleic acid molecule into cell are provided herein. As used herein, “stable integration” or “stably integrated” refers to a transfer of a nucleic acid molecule into a genome of a targeted cell that allows the targeted cell to pass the transferred nucleic acid molecule to the next generation of the transformed organism.

As used herein, “transiently introduced,” “transiently transformed,” or “transient transformation” refers to a transfer of DNA into a cell that is not transferred to the next generation of the transformed organism.

Numerous methods for transforming (e.g., providing) cells with a nucleic acid molecule or nucleoprotein complex are known in the art, which can be used according to methods of the present application. Any suitable method or technique for transformation of a cell known in the art can be used according to present methods. Non-limiting methods for transformation of cells includes polyethylene glycol-mediated transformation, biolistic transformation, liposome-mediated transfection, viral transduction, the use of one or more delivery particles, and electroporation. In an aspect, a method comprises introducing one or more nucleic acid molecules or nucleoprotein complexes to a cell using a method selected from the group consisting of polyethylene glycol-mediated transformation, biolistic transformation, liposome-mediated transfection, viral transduction, the use of one or more delivery particles, and electroporation.

In an aspect, a method comprises providing a cell with a nucleic acid molecule via polyethylene glycol-mediated transformation. In an aspect, a method comprises providing a cell with a nucleic acid molecule via biolistic transformation. In an aspect, a method comprises providing a cell with a nucleic acid molecule via liposome-mediated transfection. In an aspect, a method comprises providing a cell with a nucleic acid molecule via viral transduction. In an aspect, a method comprises providing a cell with a nucleic acid molecule via use of one or more delivery particles. In an aspect, a method comprises providing a cell with a nucleic acid molecule via microinjection. In an aspect, a method comprises providing a cell with a nucleic acid molecule via electroporation. In an aspect, a method comprises providing a cell with a nucleoprotein complex via microinjection. In an aspect, a method comprises providing a cell with a nucleoprotein complex via electroporation.

Other methods for transformation, such as vacuum infiltration, pressure, sonication, and silicon carbide fiber agitation, are also known in the art and envisioned for use with any method provided herein.

Methods of transforming cells are well known by persons of ordinary skill in the art. For instance, specific instructions for transforming plant cells by microprojectile bombardment with particles coated with nucleic acid molecules (e.g., biolistic transformation) are found in U.S. Pat. Nos. 5,550,318; 5,538,880 6,160,208; 6,399,861; and 6,153,812.

Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024.

Delivery vehicles, vectors, particles, nanoparticles, formulations and components thereof for expression of one or more elements of a nucleic acid molecule are as used in WO 2014/093622. In an aspect, a method of providing a nucleic acid molecule or a protein to a cell comprises delivery via a delivery particle. In an aspect, a method of providing a nucleic acid molecule to a cell comprises delivery via a delivery vesicle. In an aspect, a delivery vesicle is selected from the group consisting of an exosome and a liposome. In an aspect, a method of providing a nucleic acid molecule to a cell comprises delivery via a viral vector. In an aspect, a viral vector is selected from the group consisting of an adenovirus vector, a lentivirus vector, and an adeno-associated viral vector. In another aspect, a method providing a nucleic acid molecule to a cell comprises delivery via a nanoparticle. In an aspect, a method providing a nucleic acid molecule to a cell comprises microinjection. In an aspect, a method providing a nucleic acid molecule to a cell comprises polycations. In an aspect, a method providing a nucleic acid molecule to a cell comprises a cationic oligopeptide.

In an aspect, a delivery particle is selected from the group consisting of an exosome, an adenovirus vector, a lentivirus vector, an adeno-associated viral vector, a nanoparticle, a polycation, and a cationic oligopeptide. In an aspect, a method provided herein comprises the use of one or more delivery particles. In another aspect, a method provided herein comprises the use of two or more delivery particles. In another aspect, a method provided herein comprises the use of three or more delivery particles.

Suitable agents to facilitate transfer of nucleic acid molecules into a cell include agents that increase permeability of the cell to oligonucleotides or polynucleotides. Such agents to facilitate transfer of the composition into a cell include a chemical agent, or a physical agent, or combinations thereof. Chemical agents for conditioning includes (a) surfactants, (b) organic solvents, aqueous solutions, or aqueous mixtures of organic solvents, (c) oxidizing agents, (e) acids, (f) bases, (g) oils, (h) enzymes, or combinations thereof.

Agents for laboratory conditioning of a cell to permeation by polynucleotides include, e.g., application of a chemical agent, enzymatic treatment, heating or chilling, treatment with positive or negative pressure, or ultrasound treatment.

In an aspect, this disclosure provides a method of editing at least one eukaryotic cell, the method comprising: (a) introducing (i) a MAD2019-H848A variant polypeptide or a nucleic acid molecule encoding the MAD2019-H848A variant polypeptide to the at least one eukaryotic cell; and (ii) a guide RNA or a nucleic acid molecule encoding the guide RNA to the at least one eukaryotic cell, where the guide RNA comprises a nucleic acid sequence that is complementary to a target nucleic acid molecule within a genome of the eukaryotic cell; where the MAD2019-H848A variant polypeptide and the guide RNA form a nucleoprotein complex within the at least one eukaryotic cell, where the nucleoprotein complex cleaves one strand of the target nucleic acid molecule, and where at least one edit is made within the target nucleic acid molecule as compared to a control version of the target nucleic acid molecule; and (b) identifying at least one eukaryotic cell comprising the at least one edit. In an aspect, step (a) of the method further comprises introducing at least one homology arm, or a nucleic acid molecule encoding the homology arm, to the eukaryotic cell, where the at least one homology arm comprises a nucleic acid sequence comprising the at least one edit.

In an aspect, this disclosure provides a method of editing at least one eukaryotic cell, the method comprising: (a) introducing (i) a fusion protein or a nucleic acid molecule encoding the fusion protein to the at least one eukaryotic cell; and (ii) a guide RNA or a nucleic acid molecule encoding the guide RNA to the at least one eukaryotic cell, where the guide RNA comprises a nucleic acid sequence that is complementary to a target nucleic acid molecule within a genome of the eukaryotic cell; where the fusion protein and the guide RNA form a nucleoprotein complex within the at least one eukaryotic cell, where the nucleoprotein complex cleaves one strand of the target nucleic acid molecule, and where at least one edit is made within the target nucleic acid molecule as compared to a control version of the target nucleic acid molecule; and (b) identifying at least one eukaryotic cell comprising the at least one edit. In an aspect, step (a) of the method further comprises introducing at least one homology arm, or a nucleic acid molecule encoding the homology arm, to the eukaryotic cell, where the at least one homology arm comprises a nucleic acid sequence comprising the at least one edit.

The following examples of non-limiting embodiments are specifically envisioned:

1. A MAD2019-H848A variant polypeptide comprising an amino acid sequence at least 90% identical or similar to SEQ ID NO: 1, wherein the MAD2019-H848A variant polypeptide comprises an alanine at position 848 according to SEQ ID NO: 1.

2. The MAD2019-H848A variant polypeptide of embodiment 1, wherein the MAD2019-H848A variant polypeptide comprises a V1143T amino acid substitution as compared to SEQ ID NO: 1.

3. The MAD2019-H848A variant polypeptide of embodiment 1, wherein the MAD2019-H848A variant polypeptide comprises an L500R amino acid substitution, a D700A amino acid substitution, a D701P amino acid substitution, a K720S amino acid substitution, an I1142K amino acid substitution, and a V1143T amino acid substitution as compared to SEQ ID NO: 1.

4. The MAD2019-H848A variant polypeptide of embodiment 1, wherein the MAD2019-H848A variant polypeptide comprises an L500R amino acid substitution, a D700A amino acid substitution, a D701P amino acid substitution, a K720S amino acid substitution, and a V1143T amino acid substitution as compared to SEQ ID NO: 1.

5. The MAD2019-H848A variant polypeptide of embodiment 1, wherein the MAD2019-H848A variant polypeptide comprises an L500K amino acid substitution and a V1143T amino acid substitution as compared to SEQ ID NO: 1.

6. The MAD2019-H848A variant polypeptide of embodiment 1, wherein the MAD2019-H848A variant polypeptide comprises an S409R amino acid substitution, an L500K amino acid substitution, and V1143T amino acid substitution as compared to SEQ ID NO: 1.

7. The MAD2019-H848A variant polypeptide of embodiment 1, wherein the MAD2019-H848A variant polypeptide comprises a V1143T amino acid substitution and an A1221H amino acid substitution as compared to SEQ ID NO: 1.

8. The MAD2019-H848A variant polypeptide of embodiment 1, wherein the MAD2019-H848A variant polypeptide comprises an L500K amino acid substitution, a V1143T amino acid substitution, and an A1221H amino acid substitution as compared to SEQ ID NO: 1.

9. The MAD2019-H848A variant polypeptide of embodiment 1, wherein the MAD2019-H848A variant polypeptide comprises an L500K amino acid substitution, an I1142R amino acid substitution, a V1143T amino acid substitution, and an A1221H amino acid substitution as compared to SEQ ID NO: 1.

10. The MAD2019-H848A variant polypeptide of embodiment 1, wherein the MAD2019-H848A variant polypeptide comprises an L500K amino acid substitution, a D1139N amino acid substitution, a V1143T amino acid substitution, and an A1221H amino acid substitution as compared to SEQ ID NO: 1.

11. The MAD2019-H848A variant polypeptide of embodiment 1, wherein the MAD2019-H848A variant polypeptide comprises an L500K amino acid substitution, a V1143T amino acid substitution, an A1221H amino acid substitution, and a K1285R amino acid substitution as compared to SEQ ID NO: 1.

12. The MAD2019-H848A variant polypeptide of embodiment 1, wherein the MAD2019-H848A variant polypeptide comprises an amino acid substitution selected from the group consisting of: T67G, S409R, L500K, L500R, G578F, L624Q, N669S, D700A, D701P, D701N, D701T, K720S, L1110R, D1139N, I1142R, I1142K, V1143T, A1221H, K1285R, A1321R, A1321K, S1136Q, and A1139R as compared to SEQ ID NO: 1.

13. The MAD2019-H848A variant polypeptide of any one of embodiments 1 to 12, wherein the MAD2019-H848A variant polypeptide further comprises at least one nuclear localization signal.

14. A nucleic acid sequence encoding the MAD2019-H848A variant polypeptide of any one of embodiments 1 to 13.

15. A nucleoprotein complex comprising the MAD2019-H848A variant polypeptide of any one of embodiments 1 to 13 and a nucleic acid molecule.

16. The nucleoprotein complex of embodiment 15, wherein the nucleic acid molecule comprises (a) a guide; (b) a homology arm; or (c) a guide and a homology arm.

17. The nucleoprotein complex of embodiment 15 or 16, wherein the nucleic acid molecule is an RNA molecule.

18. A eukaryotic cell comprising the MAD2019-H848A variant polypeptide of any one of embodiments 1 to 13.

19. A eukaryotic cell comprising the nucleic acid sequence of embodiment 14.

20. A eukaryotic cell comprising the nucleoprotein complex of any one of embodiments 15 to 17.

21. A fusion protein comprising the MAD2019-H848A variant polypeptide of any one of embodiments 1 to 12.

22. The fusion protein of embodiment 21, wherein the fusion protein further comprises a reverse transcriptase.

23. The fusion protein of embodiment 22, wherein the reverse transcriptase is a Tf1 reverse transcriptase comprising an amino acid sequence at least 90% identical or similar to SEQ ID NO: 12.

24. The fusion protein of embodiment 23, wherein the Tf1 reverse transcriptase comprises a D362N amino acid substitution as compared to SEQ ID NO: 12.

25. The fusion protein of embodiment 23, wherein the Tf1 reverse transcriptase comprises SEQ ID NO: 13.

26. The fusion protein of embodiment 22, wherein the reverse transcriptase is derived from a reverse transcriptase selected from the group consisting of an HIV-1 (human immunodeficiency virus) reverse transcriptase, an M-MLV (Moloney murine leukemia virus) reverse transcriptase, and an AMV (avian myeloblastosis virus) reverse transcriptase.

27. The fusion protein of any one of embodiments 21 to 26, wherein the fusion protein further comprises at least one nuclear localization signal.

28. The fusion protein of any one of embodiments 21 to 27, wherein the fusion protein comprises a linker amino acid sequence.

29. The fusion protein of embodiment 22, wherein the fusion protein comprises a linker amino acid sequence positioned between the MAD2019-H848A variant polypeptide and the reverse transcriptase.

30. A fusion protein comprising a Tf1 reverse transcriptase comprising an amino acid sequence at least 90% identical or similar to SEQ ID NO: 12.

31. The fusion protein of embodiment 30, wherein the Tf1 reverse transcriptase comprises a D362N amino acid substitution as compared to SEQ ID NO: 12.

32. The fusion protein of embodiment 30, wherein the Tf1 reverse transcriptase comprises SEQ ID NO: 13.

33. The fusion protein of any one of embodiments 30 to 32, wherein the fusion protein further comprises a nickase.

34. The fusion protein of embodiment 33, wherein the nickase is a CRISPR-Cas nickase.

35. The fusion protein of embodiment 34, wherein the CRISPR-Cas nickase is a type I CRISPR-Cas nickase.

36. The fusion protein of embodiment 34, wherein the CRISPR-Cas nickase is a type II CRISPR-Cas nickase.

37. The fusion protein of embodiment 34, wherein the CRISPR-Cas nickase is a type III CRISPR-Cas nickase.

38. The fusion protein of embodiment 34, wherein the CRISPR-Cas nickase is a type IV CRISPR-Cas nickase.

39. The fusion protein of embodiment 34, wherein the CRISPR-Cas nickase is a type V CRISPR-Cas nickase.

40. The fusion protein of embodiment 34, wherein the CRISPR-Cas nickase is a type VI CRISPR-Cas nickase.

41. The fusion protein of embodiment 34, wherein the CRISPR-Cas nickase is a Cas9 nickase.

42. The fusion protein of embodiment 34, wherein the CRISPR-Cas nickase is a MAD2019 polypeptide.

43. The fusion protein of embodiment 34, wherein the CRISPR-Cas nickase is a MAD2019-H848A polypeptide.

44. The fusion protein of embodiment 34, wherein the CRISPR-Cas nickase is a MAD2019-H848A variant polypeptide.

45. The fusion protein of any one of embodiments 30 to 32, wherein the fusion protein further comprises a MAD2019-H848A variant polypeptide comprising an amino acid sequence at least 90% identical or similar to SEQ ID NO: 1, wherein the MAD2019-H848A nickase comprises an alanine at position 848 according to SEQ ID NO: 1.

46. The fusion protein of embodiment 45, wherein the MAD2019-H848A variant polypeptide comprises a V1143T amino acid substitution as compared to SEQ ID NO: 1.

47. The fusion protein of embodiment 45, wherein the MAD2019-H848A variant polypeptide comprises an L500R amino acid substitution, a D700A amino acid substitution, a D701P amino acid substitution, a K720S amino acid substitution, an I1142K amino acid substitution, and a V1143T amino acid substitution as compared to SEQ ID NO: 1.

48. The fusion protein of embodiment 45, wherein the MAD2019-H848A variant polypeptide comprises an L500R amino acid substitution, a D700A amino acid substitution, a D701P amino acid substitution, a K720S amino acid substitution, and a V1143T amino acid substitution as compared to SEQ ID NO: 1.

49. The fusion protein of embodiment 45, wherein the MAD2019-H848A variant polypeptide comprises an L500K amino acid substitution and a V1143T amino acid substitution as compared to SEQ ID NO: 1.

50. The fusion protein of embodiment 45, wherein the MAD2019-H848A variant polypeptide comprises an S409R amino acid substitution, an L500K amino acid substitution, and V1143T amino acid substitution as compared to SEQ ID NO: 1.

51. The fusion protein of embodiment 45, wherein the MAD2019-H848A variant polypeptide comprises a V1143T amino acid substitution and an A1221H amino acid substitution as compared to SEQ ID NO: 1.

52. The fusion protein of embodiment 45, wherein the MAD2019-H848A variant polypeptide comprises an L500K amino acid substitution, a V1143T amino acid substitution, and an A1221H amino acid substitution as compared to SEQ ID NO: 1.

53. The fusion protein of embodiment 45, wherein the MAD2019-H848A variant polypeptide comprises an L500K amino acid substitution, an I1142R amino acid substitution, a V1143T amino acid substitution, and an A1221H amino acid substitution as compared to SEQ ID NO: 1.

54. The fusion protein of embodiment 45, wherein the MAD2019-H848A variant polypeptide comprises an L500K amino acid substitution, a D1139N amino acid substitution, a V1143T amino acid substitution, and an A1221H amino acid substitution as compared to SEQ ID NO: 1.

55. The fusion protein of embodiment 45, wherein the MAD2019-H848A variant polypeptide comprises an L500K amino acid substitution, a V1143T amino acid substitution, an A1221H amino acid substitution, and a K1285R amino acid substitution as compared to SEQ ID NO: 1.

56. The fusion protein of embodiment 45, wherein the MAD2019-H848A variant polypeptide comprises an amino acid substitution selected from the group consisting of: T67G, S409R, L500K, L500R, G578F, L624Q, N669S, D700A, D701P, D701N, D701T, K720S, L1110R, D1139N, I1142R, I1142K, V1143T, A1221H, K1285R, A1321R, A1321K, S1136Q, and A1139R as compared to SEQ ID NO: 1.

57. The fusion protein of any one of embodiments 30 to 56, wherein the fusion protein further comprises at least one nuclear localization signal.

58. The fusion protein of any one of embodiments 30 to 57, wherein the fusion protein comprises a linker amino acid sequence.

59. The fusion protein of any one of embodiments 45 to 56, wherein the fusion protein comprises a linker amino acid sequence positioned between the MAD2019-H848A variant polypeptide and the Tf1 reverse transcriptase.

60. The fusion protein of embodiment 33, wherein the fusion protein comprises a linker amino acid sequence positioned between the nickase and the Tf1 reverse transcriptase.

61. A nucleic acid sequence encoding the fusion protein of any one of embodiments 21 to 60.

62. A nucleoprotein complex comprising the fusion protein of any one of embodiments 21 to 60 and a nucleic acid molecule.

63. The nucleoprotein complex of embodiment 62, wherein the nucleic acid molecule comprises (a) a guide; (b) a homology arm; or (c) a guide and a homology arm.

64. The nucleoprotein complex of embodiment 62 or 63, wherein the nucleic acid molecule is an RNA molecule.

65. A eukaryotic cell comprising the fusion protein of any one of embodiments 21 to 60.

66. A eukaryotic cell comprising the nucleic acid sequence of embodiment 61.

67. A eukaryotic cell comprising the nucleoprotein complex of any one of embodiments 62 to 64.

68. A method of providing a MAD2019-H848A variant polypeptide to a cell, the method comprising:
(a) obtaining a cell; and
(b) providing the cell with the MAD2019-H848A variant polypeptide of any one of embodiments 1 to 13, or a nucleic acid molecule encoding the MAD2019-H848A variant polypeptide.

69. A method of providing a fusion protein to a cell, the method comprising:
(a) obtaining a cell; and
(b) providing the cell with the fusion protein of any one of embodiments 21 to 60, or a nucleic acid molecule encoding the fusion protein.

70. The method of embodiment 68 or 69, wherein the method further comprises transfecting the cell with a nucleic acid molecule encoding (a) a guide; (b) a homology arm; or (c) a guide and a homology arm.

71. The method of embodiment 69, wherein the guide is an RNA molecule that forms a nucleoprotein complex with the MAD2019-H848A variant polypeptide within the cell.

72. The method of embodiment 71, wherein the guide comprises a sequence complementary to a target nucleic acid molecule within the cell.

73. The method of embodiment 72, wherein the nucleoprotein complex edits the target nucleic acid molecule within the cell.

74. The method of embodiment 70, wherein the guide is an RNA molecule that forms a nucleoprotein complex with the fusion protein within the cell.

75. The method of embodiment 74, wherein the guide comprises a sequence complementary to a target nucleic acid molecule within the cell.

76. The method of embodiment 75, wherein the nucleoprotein complex edits the target nucleic acid molecule within the cell.

77. The method of any one of embodiments 72, 73, 75, and 76, wherein the target nucleic acid molecule is positioned within a nuclear genome of the cell.

78. The method of any one of embodiments 72, 73, 75, and 76, wherein the target nucleic acid molecule is positioned within a mitochondrial genome of the cell.

79. The method of any one of embodiments 68 to 76, wherein the providing comprises a method selected from the group consisting of polyethylene glycol-mediated transformation, biolistic transformation, liposome-mediated transfection, viral transduction, the use of one or more delivery particles, microinjection, and electroporation.

80. A method of editing at least one eukaryotic cell, the method comprising:
(a) introducing
   (i) the MAD2019-H848A variant polypeptide of any one of embodiments 1 to 13, or a nucleic acid molecule encoding the MAD2019-H848A variant polypeptide to the at least one eukaryotic cell; and
   (ii) a guide RNA or a nucleic acid molecule encoding the guide RNA to the at least one eukaryotic cell, wherein the guide RNA comprises a nucleic acid sequence that is complementary to a target nucleic acid molecule within a genome of the eukaryotic cell;
      wherein the MAD2019-H848A variant polypeptide and the guide RNA form a nucleoprotein complex within the at least one eukaryotic cell, wherein the nucleoprotein complex cleaves one strand of the target nucleic acid molecule, and wherein at least one edit is made within the target nucleic acid molecule as compared to a control version of the target nucleic acid molecule; and
(b) identifying at least one eukaryotic cell comprising the at least one edit.

81. A method of editing at least one eukaryotic cell, the method comprising:
(a) introducing
   (i) the fusion protein of any one of embodiments 21 to 60, or a nucleic acid molecule encoding the fusion protein to the at least one eukaryotic cell; and
   (ii) a guide RNA or a nucleic acid molecule encoding the guide RNA to the at least one eukaryotic cell, wherein the guide RNA comprises a nucleic acid sequence that is complementary to a target nucleic acid molecule within a genome of the eukaryotic cell;
      wherein the fusion protein and the guide RNA form a nucleoprotein complex within the at least one eukaryotic cell, wherein the nucleoprotein complex cleaves one strand of the target nucleic acid molecule, and wherein at least one edit is made within the target nucleic acid molecule as compared to a control version of the target nucleic acid molecule; and
(b) identifying at least one eukaryotic cell comprising the at least one edit.

82. The method of embodiment 80 or 81, wherein step (a) of the method further comprises introducing at least one homology arm, or a nucleic acid molecule encoding the homology arm, to the eukaryotic cell, and wherein the at least one homology arm comprises a nucleic acid sequence comprising the at least one edit.

83. The method of any one of embodiments 80 to 82, wherein the at least one edit comprises an insertion.

84. The method of any one of embodiments 80 to 82, wherein the at least one edit comprises a deletion.

85. The method of any one of embodiments 80 to 82, wherein the at least one edit comprises a substitution.

86. The method of any one of embodiments 80 to 82, wherein the at least one edit comprises a substitution of a single nucleotide.

87. The method of embodiment 86, wherein the substitution of a single nucleotide comprises a transition.

88. The method of embodiment 86, wherein the substitution of a single nucleotide comprises a transversion.

89. A guide RNA (gRNA) comprising a scaffold region having a nucleic acid sequence at least 80% identical to SEQ ID NO: 24.

90. The gRNA of embodiment 89, wherein the nucleic acid sequence is at least 90% identical to SEQ ID NO: 24.

91. The gRNA of embodiment 89, wherein the nucleic acid sequence is at least 95% identical to SEQ ID NO: 24.

92. The gRNA of embodiment 89, wherein the nucleic acid sequence is 100% identical to SEQ ID NO: 24.

93. A nucleoprotein complex comprising a fusion protein and the gRNA of any one of embodiments 89 to 92.

94. The nucleoprotein complex of embodiment 93, wherein the fusion protein comprises a MAD2019-H848A variant polypeptide.

95. The nucleoprotein complex of embodiment 93 or 94, wherein the fusion protein comprises a reverse transcriptase.

96. A nucleoprotein complex comprising a MAD2019-H848A variant polypeptide and the gRNA of any one of embodiments 89 to 92.

97. A nucleoprotein complex comprising a nickase and the gRNA of any one of embodiments 89 to 92.

98. A nucleoprotein complex comprising the gRNA of any one of embodiments 89 to 92.

Having now generally described the disclosure, the same will be more readily understood through reference to the following examples that are provided by way of illustration, and are not intended to be limiting of the present disclosure, unless specified.

### EXAMPLES

### Example 1. Workflow Overview

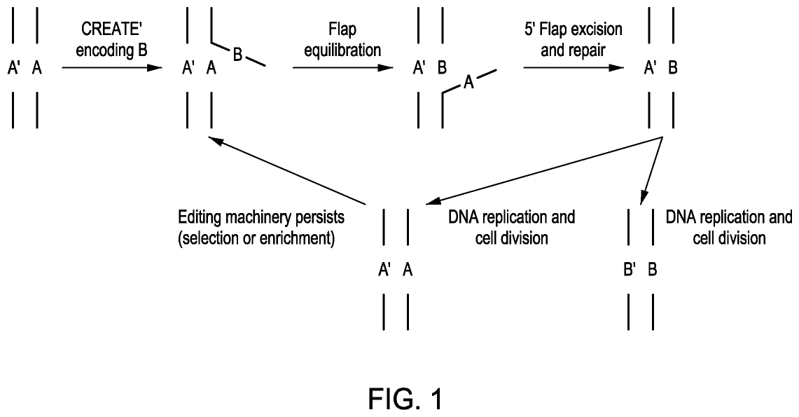

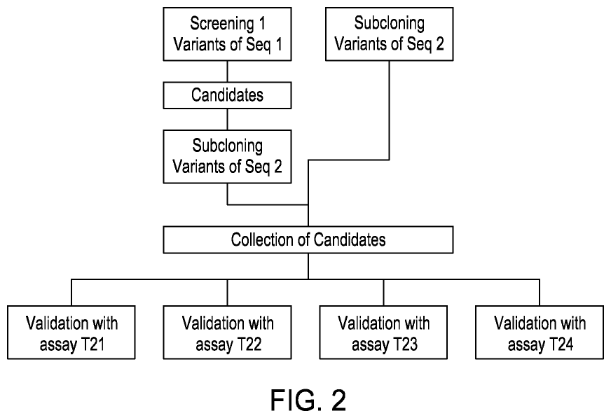

The MAD2019 variant polypeptides provided herein, based on MAD2019-H848A (SEQ ID NO: 1) were identified using the method depicted in FIG. 2. First, pools of variants (Seq 1 in FIG. 2; Seq 1 refers to a SEQ ID NO: 1 variant that has a histidine at amino acid position 848 instead of an alanine) were generated by site saturation mutagenesis for all residues of SEQ ID NO: 1. Another pool of variants of Seq 1 with H848 were generated by site saturation mutagenesis for all residues. Next, screening was performed to identify SEQ ID NO: 1 variants having improved cutting efficiency as compared to SEQ ID NO: 1 a fusion protein comprising Seq 1 (screening 1 in FIG. 2) or to identify variants having improved CREATE Fusion editing efficiency (screening 2 in FIG. 2) as compared to Seq 1 or a fusion protein comprising Seq 1. See, for example, U.S. Pat. No. 11,268,078.

Following identification of candidates for improved cutting efficiency, the variants are introduced into MAD2019-H848A (FIG. 2) for further evaluation. A second round of screening to identify MAD2019-H848A variant polypeptides with improved CREATE fusion editing generated by site saturation mutagenesis for all residues. Pools of candidates from both screenings (Collection of Candidates in FIG. 2) are further validated to measure improvement in CREATE fusion editing via multiple assays (e.g., validations). Table 2 provides a list of MAD2019-H848A variants that exhibited improved CREATE fusion editing as compared to MAD2019-H848A in at least one assay (e.g., assay T21, assay T22, assay T23, assay T24 in FIG. 2).

Screening for cutting efficiency involves detecting the fluorescence generated from expression of a single copy of a synthetic GREEN FLUORESCENCE PROTEIN (GFP) integrated randomly the genome of a cell. If no cutting is performed, the GFP signal will be similar to a GFP expression from a control cell when grown under similar conditions.

If a double-stranded break is generated, which interrupts normal expression or activity of GFP, a reduced GFP signal will be observed. However, if a precise edit is made, GFP can be converted to emit a blue wavelength of fluorescence (BFP) that can be detected and quantified. See, for example, Heim et al., Proc. Natl. Sci. USA, 91:12501-12504 (1994); and Glaser et al., Mol. Ther. Nucleic Acids, 5: e334 (2016).

TABLE 2
MAD2019-H848A variant polypeptides with improved CREATE fusion
editing in at least one assay as compared to MAD2019-H848A (SEQ
ID NO: 1). The MAD2019-H848A polypeptide variant column denotes
the amino acid residue change and position of the change for each
variant. For example, T67G refers to a change from threonine (T)
to glycine (G) at position 67 of SEQ ID NO: 1. Values in the Assay
columns refer to fold activity as compared to SEQ ID NO: 1, where
a value of 1.0 refers to equal activity to SEQ ID NO: 1. Assay
refers to the guide sequences provided in Table 3.

| | |
|---|---|
| MAD2019H848A | |
| polypeptide | Assay |

| variant | T21 | T22 | T23 | T24 |
|---|---|---|---|---|
| T67G | 1.0 | 1.2 | 1.1 | 1.1 |
| S409R | 0.9 | 1.2 | 1.1 | 0.8 |
| L500K | 1.3 | 1.2 | 1.1 | 1.1 |
| L500R | 1.2 | 1.2 | 1.1 | 1.1 |
| G578F | 1.2 | 1.2 | 1.1 | 1.0 |
| L624Q | 1.3 | 1.3 | 1.0 | 0.9 |
| N669S | 1.1 | 1.2 | 1.0 | 1.1 |
| D700A | 1.6 | 1.2 | 1.0 | 1.1 |
| D701P | 1.5 | 1.2 | 1.0 | 1.1 |
| D701N | 1.6 | 1.3 | 1.0 | 1.0 |
| D701T | 1.4 | 1.3 | 1.0 | 1.0 |
| K720S | 1.2 | 1.3 | 1.0 | 1.0 |
| L1110R | 1.2 | 1.1 | 1.1 | 1.0 |
| D1139N | 1.4 | 1.1 | 1.1 | 0.9 |
| I1142R | 1.0 | 1.1 | 1.0 | 0.9 |
| I1142K | 1.2 | 1.0 | 1.1 | 0.8 |
| V1143T | 1.6 | 1.1 | 1.1 | 0.9 |
| A1221H | 1.2 | 1.2 | 1.1 | 1.0 |
| K1285R | 1.1 | 1.4 | 1.0 | 1.0 |
| A1321R | 1.5 | 1.2 | 1.1 | 1.0 |
| A1321K | 1.3 | 1.2 | 1.1 | 0.9 |
| S1336Q | 1.1 | 1.0 | 1.0 | 1.2 |
| A1339R | 1.2 | 1.2 | 1.0 | 1.0 |

### Example 2. Vector Construct for MADzymes and Guide-Expressing Plasmids

MAD2019 variant polypeptides were cloned under the control of a CMV promoter to be expressed in HEK293T (human kidney) cells. MAD2019 variant polypeptides were also cloned under the EF1-alpha promoter to express in induced pluripotent stem cells (PGP168). The HEK293T cell line comprised a single copy of a synthetic GREEN FLUORESCENCE PROTEIN (GFP) integrated randomly in its genome. For CREATE fusion editing assays, the MAD2019 polypeptide and MAD2019-H848A polypeptide variants were fused with a reverse transcriptase and expressed as a single fusion construct.

Guide RNAs targeting the GFP locus were cloned under the control of the human U6 promoter with a single-guide RNA (sgRNA) scaffold (SEQ ID NO: 24) and a guide sequence (see Table 3) positioned at the 5′-end of each sgRNA. A CREATE fusion homology arm was positioned at the 3′-end of each sgRNA (see Table 3) if a CREATE fusion editing assay was performed with an extra RNA stabilizing sequence element (e.g., SEQ ID NO: 25) followed by a transcription terminator (e.g., 5′-TTTTTTT-3′) at the 3′-end of the final sequence.

TABLE 3
Guide and homology arm sequences
used in CREATE fusion assays. PAM
refers to protospacer adjacent motif.

| | | Homology | |
|---|---|---|---|
| | Guide | Arm | |
| | SEQ ID | SEQ ID | PAM |
| Assay | NO | NO | Sequence |
| T21 (Screening | 3 | 4 | 5′-NGG-3′ |
| 2 guide) | | | |
| (CFg17) | | | |
| T22 (CFg18) | 5 | 6 | 5′-NGG-3′ |
| T23 (CFg1) | 7 | 8 | 5′-NGG-3′ |
| T24 (CFg5) | 9 | 10 | 5′-NGG-3′ |
| Screening 1 | 11 | n/a | 5′-NAG-3′ |
| guide | | | |
| CFg25 | 26 | 27 | 5′-NGG-3′ |
| CFg791 | 28 | 29 | 5′-NAG-3′ |

### Example 3. Transfection in HEK293T Cells

A plasmid expressing a guide (50 ng), or pUC19 as a negative control, and a plasmid expressing MAD2019-H848A variant polypeptide (see Table 2) fused with M-MLV reverse transcriptase (50 ng) were mixed with 1 μL PolyFect™ Transfection Reagent and diluted in 35 μL OptiMem™. This mixture was added to 25k TrypLE singulated HEK293T host cells comprising the synthetic GFP target locus in 100 μL DMEM (Dulbecco's Modified Eagle's Medium) for reverse transfection.

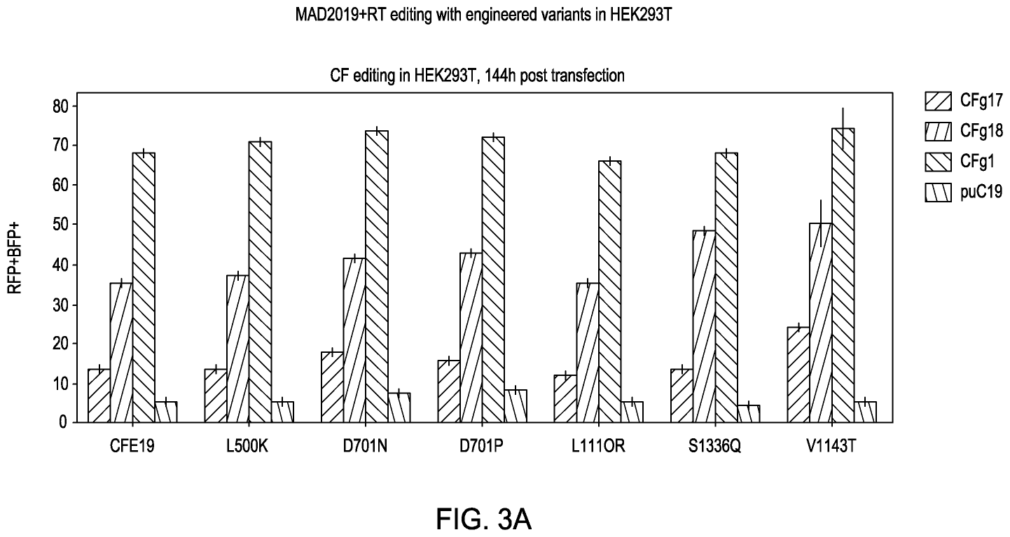

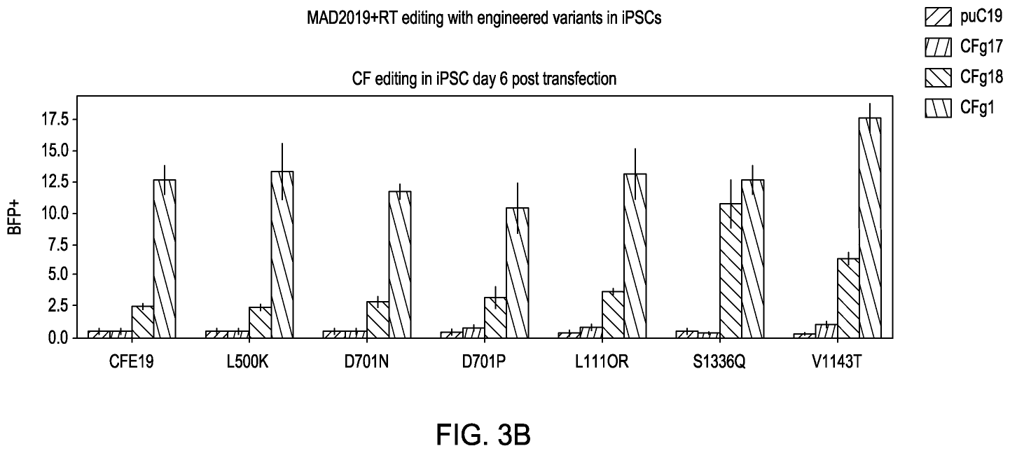

Six days after transfection, the HEK293T cells were collected and analyzed using a flow cytometer to detect depletion of the GFP signal, which is evidence of a double-strand break that interrupted expression of GFP, or to detect a BFP signal, which is evidence of precise genome editing. See. FIG. 3A.

### Example 4. Transfection in Induced Pluripotent Stem Cells

A plasmid expressing a guide (25 ng), or pUC19 as a negative control, and a plasmid expressing MAD2019-H848A variant polypeptide (see Table 2) fused with M-MLV reverse transcriptase (25 ng) were mixed 0.75 μL Lipofectamine™ Stem Transfection Reagent and diluted in OptiMem™. Forward transfection on pre-plated induced pluripotent stem cells was performed.

Six days after transfection, the induced pluripotent stem cells were collected and analyzed using a flow cytometer to detect depletion of the GFP signal, which is evidence of a double-strand break that interrupted expression of GFP, or to detect a BFP signal, which is evidence of precise genome editing. See. FIG. 3B.

### Example 5. Vector Construct for MADzymes and Guide Expressing Plasmids

Fusion proteins of MAD2019-H848A or the MAD2019-H848A polypeptide variants described in Table 2, and the Schizosaccharomyces pombe reverse transcriptase Tf1 (SEQ ID NO: 12) or the Tf1 variant Tf1-D364N (SEQ ID NO: 13) were cloned into plasmids under the control of the CMV promoter for expression in mammalian cells.

Guide RNAs targeting the GFP locus were cloned under the control of the human U6 promoter with a single-guide RNA (sgRNA) scaffold (SEQ ID NO: 14) and a guide sequence (see Table 3) positioned at the 5′-end of each sgRNA. A CREATE fusion homology arm was positioned at the 3′-end of each sgRNA (see Table 3).

The constructs are intended to be introduced into HEK293T cells that comprise a single copy of GFP randomly integrated into the genome of the cells.

### Example 6. Transfection in HEK293T Cells

Two groups of plasmid sets are prepared and transfected into HEK293T cells.

The first group comprises a plasmid expressing a guide (50 ng), or pUC19 as a negative control, and a plasmid expressing MAD2019-H848A::Tf1 (SEQ ID NOs: 1 and 12, respectively) (50 ng) are mixed with 1 μL PolyFect™ Transfection Reagent and diluted in 35 μL OptiMem™. This mixture was added to 25k TrypLE singulated HEK293T host cells comprising the synthetic GFP target locus in 100 μL DMEM for reverse transfection.

The second group comprises a plasmid expressing a guide (50 ng), or pUC19 as a negative control, and a plasmid expressing MAD2019-H848A::Tf1-D364N (SEQ ID NOs: 1 and 13, respectively) (50 ng) are mixed with 1 μL PolyFect™ Transfection Reagent and diluted in 35 μL OptiMem™. This mixture was added to 25k TrypLE singulated HEK293T host cells comprising the synthetic GFP target locus in 100 μL DMEM for reverse transfection.

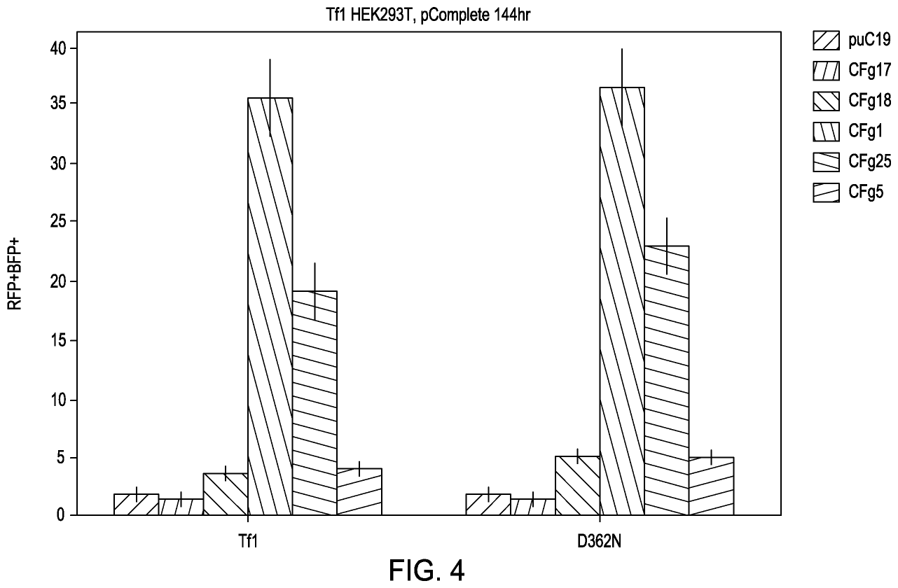

Six days after transfection, the induced pluripotent stem cells were collected and analyzed using a flow cytometer to detect depletion of the GFP signal, which is evidence of a double-strand break that interrupted expression of GFP, or to detect a BFP signal, which is evidence of precise genome editing. See. FIG. 4.

### Example 7. Additional Transfection in HEK293T Cells

Two additional groups of plasmid sets are prepared and transfected into HEK293T cells.

The first group comprises 23 unique mixtures. Each mixture comprises a plasmid expressing a guide (50 ng), or pUC19 as a negative control, and one of the 23 MAD2019-H848A polypeptide variants provided in Table 2::Tf1 (50 ng). Each of the 23 mixtures is independently mixed with 1 μL PolyFect™ Transfection Reagent and diluted in 35 μL OptiMem™. Each of the mixtures is added to a single, independent aliquot of 25k TrypLE singulated HEK293T host cells comprising the synthetic GFP target locus in 100 μL DMEM for reverse transfection, resulting in 23 unique sets of plasmid/cell combinations.

The second group comprises 23 unique mixtures. Each mixture comprises a plasmid expressing a guide (50 ng), or pUC19 as a negative control, and one of the 23 MAD2019-H848A polypeptide variants provided in Table 2::Tf1-D364N (50 ng). Each of the 23 mixtures is independently mixed with 1 μL PolyFect™ Transfection Reagent and diluted in 35 μL OptiMem™. Each of the mixtures is added to a single, independent aliquot of 25k TrypLE singulated HEK293T host cells comprising the synthetic GFP target locus in 100 μL DMEM for reverse transfection, resulting in 23 unique sets of plasmid/cell combinations.

Six days after transfection, the induced pluripotent stem cells were collected and analyzed using a flow cytometer to detect depletion of the GFP signal, which is evidence of a double-strand break that interrupted expression of GFP, or to detect a BFP signal, which is evidence of precise genome editing.

### Example 8. Generation of Additional MAD2019-H848A Variant Polypeptides

CREATE fusion enzyme 19 (CFE19) comprises a nuclear localization signal (SEQ ID NO: 15), a MAD2019-H848A nickase (SEQ ID NO: 16), a linker (SEQ ID NO: 17), a MLV reverse transcriptase (SEQ ID NO: 18) and a second nuclear localization signal (SEQ ID NO: 19). The full amino acid sequence for CFE19 is provided as SEQ ID NO: 20.

CFE19 is subjected to editing to generate combinatorial MAD2019-H848A variant polypeptides. Table 4 provides a summary of the variants produced.

TABLE 4
CFE19 variants

| | MAD2019-H848A | |
|---|---|---|
| | additional amino acid | SEQ ID NO |
| Name | substitutions | (if applicable) |
| CFE19 | — | 20 |
| CFE19v1 or VT | V1143T | 21 |
| CFE19v2 | L500R, D700A, D701P, | 22 |
| | K720S, I1142K, V1143T | |
| CFE19v3 | L500R, D700A, D701P, | 23 |
| | K720S, V1143T | |
| KT | L500K, V1143T | |
| RKT | S409R, L500K, V1143T | |
| TH | V1143T, A1221H | |
| KTH | L500K, I1142R, V1143T, | |
| | A1221H | |
| KRTH | L500K, I1142R, V1143T, | |
| | A1221H | |
| KNTH | L500K, D1139N, V1143T, | |
| | A1221H | |
| KTHR | L500K, V1143T, A1221H, | |
| | K1285R | |

Each of the CFE19 variants provided in Table 4 is expressed from a plasmid under the control of an EF1-alpha promoter after being introduced to induced pluripotent stem cells. Expression of each CFE19 variant is monitored via an mCherry reporter that is connected by a T2A linker.

Forward transfection on pre-plated induced pluripotent stem cells comprising single copy of a randomly inserted sequence encoding GFP was performed.

Six days after transfection, the induced pluripotent stem cells were collected and analyzed using a flow cytometer to detect depletion of the GFP signal, which is evidence of a double-strand break that interrupted expression of GFP, or to detect a BFP signal, which is evidence of precise genome editing. See FIG. 5 In particular, CFE19v3 exhibited overall performance improvement for multiple targets.

1-98. (canceled)

99. A fusion protein comprising a MAD2019-H848A variant polypeptide and a reverse transcriptase, wherein the MAD2019-H848A variant polypeptide comprises an amino acid sequence at least 90% identical or similar to SEQ ID NO: 1 and an alanine at position 848 according to SEQ ID NO: 1.

100. The fusion protein of claim 99, wherein the MAD2019-H848A variant polypeptide comprises a V1143T amino acid substitution as compared to SEQ ID NO: 1.

101. The fusion protein of claim 99, wherein the MAD2019-H848A variant polypeptide comprises an L500R amino acid substitution, a D700A amino acid substitution, a D701P amino acid substitution, a K720S amino acid substitution, an I1142K amino acid substitution, and a V1143T amino acid substitution as compared to SEQ ID NO: 1.

102. The fusion protein of claim 99, wherein the MAD2019-H848A variant polypeptide comprises an L500R amino acid substitution, a D700A amino acid substitution, a D701P amino acid substitution, a K720S amino acid substitution, and a V1143T amino acid substitution as compared to SEQ ID NO: 1.

103. The fusion protein of claim 99, wherein the MAD2019-H848A variant polypeptide comprises an L500K amino acid substitution and a V1143T amino acid substitution as compared to SEQ ID NO: 1.

104. The fusion protein of claim 99, wherein the MAD2019-H848A variant polypeptide comprises an S409R amino acid substitution, an L500K amino acid substitution, and V1143T amino acid substitution as compared to SEQ ID NO: 1.

105. The fusion protein of claim 99, wherein the MAD2019-H848A variant polypeptide comprises a V1143T amino acid substitution and an A1221H amino acid substitution as compared to SEQ ID NO: 1.

106. The fusion protein of claim 99, wherein the MAD2019-H848A variant polypeptide comprises an L500K amino acid substitution, a V1143T amino acid substitution, and an A1221H amino acid substitution as compared to SEQ ID NO: 1.

107. The fusion protein of claim 99, wherein the MAD2019-H848A variant polypeptide comprises an L500K amino acid substitution, an I1142R amino acid substitution, a V1143T amino acid substitution, and an A1221H amino acid substitution as compared to SEQ ID NO: 1.

108. The fusion protein of claim 99, wherein the MAD2019-H848A variant polypeptide comprises an L500K amino acid substitution, a D1139N amino acid substitution, a V1143T amino acid substitution, and an A1221H amino acid substitution as compared to SEQ ID NO: 1.

109. The fusion protein of claim 99, wherein the MAD2019-H848A variant polypeptide comprises an L500K amino acid substitution, a V1143T amino acid substitution, an A1221H amino acid substitution, and a K1285R amino acid substitution as compared to SEQ ID NO: 1.

110. The fusion protein of claim 99, wherein the MAD2019-H848A variant polypeptide comprises an amino acid substitution selected from the group consisting of: T67G, S409R, L500K, L500R, G578F, L624Q, N669S, D700A, D701P, D701N, D701T, K720S, L1110R, D1139N, I1142R, I1142K, V1143T, A1221H, K1285R, A1321R, A1321K, S1136Q, and A1139R as compared to SEQ ID NO: 1.

111. The fusion protein of claim 99, wherein the reverse transcriptase is a Tf1 reverse transcriptase comprising an amino acid sequence at least 90% identical or similar to SEQ ID NO: 12.

112. The fusion protein of claim 111, wherein the Tf1 reverse transcriptase comprises a D364N amino acid substitution as compared to SEQ ID NO: 12.

113. The fusion protein of claim 111, wherein the Tf1 reverse transcriptase comprises SEQ ID NO: 13.

114. The fusion protein of claim 99, wherein the reverse transcriptase is derived from a reverse transcriptase selected from the group consisting of an HIV-1 (human immunodeficiency virus) reverse transcriptase, an M-MLV (Moloney murine leukemia virus) reverse transcriptase, and an AMV (avian myeloblastosis virus) reverse transcriptase.

115. The fusion protein of claim 99, wherein the fusion protein further comprises at least one nuclear localization signal.

116. The fusion protein of claim 99, wherein the fusion protein further comprises a linker amino acid sequence positioned between the MAD2019-H848A variant polypeptide and the reverse transcriptase.

117. The fusion protein of claim 99, wherein the fusion protein comprises a linker amino acid sequence positioned between the MAD2019-H848A variant polypeptide and the Tf1 reverse transcriptase.

118. A nucleoprotein complex comprising the fusion protein of claim 99 and a nucleic acid molecule.

119. The nucleoprotein complex of claim 118, wherein the nucleic acid molecule comprises (a) a guide; (b) a homology arm; or (c) a guide and a homology arm.

120. A method of editing at least one eukaryotic cell, the method comprising:
(a) introducing
(i) the fusion protein of claim 99, or a nucleic acid molecule encoding the fusion protein to the at least one eukaryotic cell; and
(ii) a guide RNA or a nucleic acid molecule encoding the guide RNA to the at least one eukaryotic cell, wherein the guide RNA comprises a nucleic acid sequence that is complementary to a target nucleic acid molecule within a genome of the eukaryotic cell;
wherein the fusion protein and the guide RNA form a nucleoprotein complex within the at least one eukaryotic cell, wherein the nucleoprotein complex cleaves one strand of the target nucleic acid molecule, and wherein at least one edit is made within the target nucleic acid molecule as compared to a control version of the target nucleic acid molecule; and
(b) identifying at least one eukaryotic cell comprising the at least one edit.
